SEQUENCE LISTING (4 sequences)

SEQ ID NO: 1 - DNA, 24 bp, Artificial Sequence
  Universal priming sequence
tacaccggcgttatgcgtcgagac

SEQ ID NO: 2 - DNA, 24 bp, Artificial Sequence
  Universal priming sequence
attgcggggaccgatgatcgcgtc

SEQ ID NO: 3 - DNA, 48 bp, Artificial Sequence
  Universal primer sequence
taatgatacggcgaccaccgagatctacaccggcgttatgcgtcgaga

SEQ ID NO: 4 - DNA, 57 bp, Artificial Sequence
  Universal priming sequence
tcaagcagaagacggcatacgagatnnnnnaaacgacgcgatcatcggtccccgcaa

(19) United States
(12) Patent Application Publication
(10) Pub. No.: US 2013/0143213 A1
(43) Pub. Date: Jun. 6, 2013

(54) DETECTION OF GENETIC ABNORMALITIES

(71) Applicant: Ariosa Diagnostics, Inc., San Jose, CA (US)

(72) Inventors: Arnold Oliphant, San Jose, CA (US); Andrew Sparks, San Jose, CA (US); Ken Song, San Jose, CA (US); John Stuelpnagel, San Jose, CA (US)

(73) Assignee: ARIOSA DIAGNOSTICS, INC., San Jose, CA (US)

(21) Appl. No.: 13/689,206

(22) Filed: Nov. 29, 2012

Related U.S. Application Data
(63) Continuation of application No. 13/356,575, filed on Jan. 23, 2012.
Application No. 13/356,575 is a continuation-in-part of application No. 13/338,963, filed on Dec. 28, 2011.
Application No. 13/338,963 is a continuation-in-part of application No. 13/316,154, filed on Dec. 9, 2011.
(60) Provisional application No. 61/436,132, filed on Jan. 25, 2011.
(60) Provisional application No. 61/436,135, filed on Jan. 25, 2011.

(51) Int. Cl. C12Q 1/68

(52) U.S. Cl. CPC C12Q 1/6827
USPC 435/6.11

(57) ABSTRACT

The present invention provides assay systems and related methods for determining genetic abnormalities in mixed samples comprising cell free DNA from both normal and putative genetically atypical cells. Exemplary mixed samples for analysis using the assay systems of the invention include samples comprising both maternal and fetal cell free DNA and samples that contain DNA from normal cells and circulating cancerous cells.

### CROSS-REFERENCE TO RELATED APPLICATIONS

The present application is a continuation of U.S. Ser. No. 13/356,575, filed Jan. 23, 2012, which is a continuation-in-part of U.S. Ser. No. 13/338,963, filed Dec. 28, 2011; which is a continuation-in-part of U.S. Ser. No. 13/316,154, filed Dec. 9, 2011; which claims priority to U.S. Ser. No. 61/436,132, filed Jan. 25, 2011 and U.S. Ser. No. 61/436,135, filed Jan. 25, 2011, all of which are herein incorporated by reference in their entirety.

### FIELD OF THE INVENTION

This invention relates to diagnosis of genetic abnormalities and assay systems for such diagnosis.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Genetic abnormalities account for a wide number of pathologies, including pathologies caused by chromosomal aneuploidy (e.g., Down syndrome), germline mutations in specific genes (e.g., sickle cell anemia), and pathologies caused by somatic mutations (e.g., cancer). Diagnostic methods for determining such genetic anomalies have become standard techniques for identifying specific diseases and disorders, as well as providing valuable information on disease source and treatment options.

For example, prenatal screening and diagnosis are routinely offered in antenatal care and are considered to be important in allowing women to make informed choices about pregnancies affected by genetic conditions. Conventional methods of prenatal diagnostic testing currently requires removal of a sample of fetal cells directly from the uterus for genetic analysis, using either chorionic villus sampling (CVS) typically between 11 and 14 weeks gestation or amniocentesis typically after 15 weeks. However, these invasive procedures carry a risk of miscarriage of around 1%. Mujezinovic and Alfirevic, Obstet Gynecol 2007:110:687-694.

Although these approaches to obtaining fetal DNA currently provide the gold standard test for prenatal diagnosis, many women decide not to undergo invasive testing, primarily because it is unpleasant and carries a small but significant risk of miscarriage. A reliable and convenient method for non-invasive prenatal diagnosis has long been sought to reduce this risk of miscarriage and allow earlier testing. Although some work has investigated using fetal cells obtained from the cervical mucus (Fejgin M D et al., Prenat Diagn 2001; 21:619-62.1; Mantzaris et al., ANZJOG 2005; 45:529-532), most research has focused on strategies for detecting genetic elements from the fetus present in the maternal circulation. It has been demonstrated that there is bidirectional traffic between the fetus and the mother during pregnancy (Lo et al., Blood 1996; 88; 4390-4395), and multiple studies have shown that both intact fetal cells and cell-free fetal nucleic acids cross the placenta and circulate in the maternal bloodstream (See, e.g., Chiu R W and Lo Y M, Semin Fetal Neonatal Med. 2010 Nov. 11).

In particular, more recent attempts to identify aneuploidies have used maternal blood as a starting material. Such efforts have included the use of cell free DNA to detect fetal aneuploidy in a sample from a pregnant female, including use of massively parallel shotgun sequencing (MPSS) to quantify precisely the increase in cfDNA fragments from trisomic chromosomes. The chromosomal dosage resulting from fetal aneuploidy, however, is directly related to the fraction of fetal cfDNA. Variation of fetal nucleic acid contribution between samples can thus complicate the analysis, as the level of fetal contribution to a maternal sample will vary the amounts needed to be detected for calculating the risk that a fetal chromosome is aneuploid.

For example, a cfDNA sample containing 4% DNA from a fetus with trisomy 21 should exhibit a 2% increase in the proportion of reads from chromosome 21 (chr21) as compared to a normal fetus. Distinguishing a trisomy 21 from a normal fetus with high confidence using a maternal sample with a fetal nucleic acid percentage of 4% requires a large number (>93K) of chromosome 21 observations, which is challenging and not cost-effective using non-selective techniques such as MPSS.

There is thus a need for non-invasive methods of screening for genetic abnormalities, including aneuploidies, in mixed samples comprising normal and putative abnormal DNA. The present invention addresses this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present invention provides assay systems and related methods for determining genetic abnormalities in mixed samples comprising genomic material (e.g., cell free DNA) from normal and putative genetically atypical cells or tissues from an individual, e.g., a pregnant woman. Exemplary mixed samples for analysis using the assay systems of the invention include patient samples comprising both maternal and fetal cell free DNA and samples that contain cell free DNA from normal cells and circulating cancerous cells.

In one aspect, the assay system utilizes enrichment and detection of selected nucleic acid regions in cell free DNA in a mixed sample to identify the presence or absence of a chromosomal aneuploidy. Levels of selected nucleic acid regions can be determined for a genomic region of interest (e.g., a chromosome or a portion thereof) and compared to the quantities of nucleic acid regions of one or more other genomic regions of interest and/or one or more reference genomic regions to detect potential aneuploidies based on chromosome frequencies in the mixed sample.

In a particular aspect, the invention provides a method comprising 1) providing a mixed sample; 2) isolating selected genomic regions from the mixed sample; 3) amplifying the selected genomic regions using one or more rounds of amplification; and 4) detecting the selected genomic regions by sequence determination. In some aspects, the isolation of the genomic region involves an initial selective amplification step. In other aspects, the isolation of the genomic region comprises a hybridization step.

In one general aspect, the invention provides an assay system for detection of a copy number variation in a genomic region of interest in a mixed sample, comprising the steps of providing a mixed sample comprising cell free DNA, enriching for two or more selected nucleic acid regions from a first genomic region of interest in the mixed sample; enriching for two or more selected nucleic acid regions from a second genomic region of interest in the mixed sample, determining the relative frequency of the selected nucleic acid regions from the first and second genomic regions of interest, comparing the relative frequency of the selected nucleic acid regions from the first and second genomic region of interest, and identifying the presence or absence of a copy number variation based on the compared relative frequencies.

In another aspect, the invention provides an assay system for detection of the presence or absence of an aneuploidy, comprising the steps of providing a mixed sample comprising cell free DNA originating from normal and putatively abnormal cells or tissue, enriching for two or more selected nucleic acid regions from a first chromosome of interest in the mixed sample; enriching for two or more selected nucleic acid regions from a second chromosome of interest in the mixed sample, determining the relative frequency of the selected nucleic acid regions from the first and second chromosomes of interest, comparing the relative frequency of the selected nucleic acid regions from the first and second chromosomes of interest, and identifying the presence or absence of an aneuploidy based on the compared relative frequencies.

In one specific aspect, the invention provides an assay system for detection of the presence or absence of a fetal chromosomal abnormality comprising the steps of providing a mixed sample comprising cell free DNA, isolating two or more selected non-polymorphic nucleic acid regions from a first genomic region of interest in the mixed sample, isolating two or more selected non-polymorphic nucleic acid regions from a second genomic region of interest in the mixed sample, amplifying the selected nucleic acid regions from the first and second genomic regions using one or more rounds of amplification, detecting the amplified nucleic acid regions, quantifying the relative frequency of the selected nucleic acid regions from the first and second genomic regions of interest, comparing the relative frequency of the selected nucleic acid regions from the first and second genomic regions of interest; and identifying the presence or absence of a fetal chromosomal abnormality based on the compared relative frequency. In some aspects, the detected chromosomal abnormality is an insertion or duplication. In other aspects, the chromosomal abnormality is an aneuploidy.

In another specific aspect, the invention provides an assay system for detection of the presence or absence of a fetal aneuploidy comprising the steps of providing a mixed sample comprising cell free DNA, isolating two or more selected non-polymorphic nucleic acid regions from a first chromosome of interest in the mixed sample, isolating two or more selected non-polymorphic nucleic acid regions from a second chromosome of interest in the mixed sample, amplifying the selected nucleic acid regions from the first and second chromosomes using one or more rounds of amplification, detecting the amplified nucleic acid regions, quantifying the relative frequency of the selected nucleic acid regions from the first and second chromosomes of interest, comparing the relative frequency of the selected nucleic acid regions from the first and second chromosomes of interest, and identifying the presence or absence of an aneuploidy based on the compared relative frequencies of the first and second chromosome of interest.

In yet another specific aspect, the invention provides an assay system for detection of the presence or absence of an aneuploidy, comprising the steps of providing a maternal sample comprising maternal and fetal cell free DNA, selectively amplifying two or more nucleic acid regions from a first chromosome of interest in the maternal sample, selectively amplifying two or more nucleic acid regions from a second chromosome of interest in the maternal sample, detecting the amplified nucleic acid regions, quantifying the relative frequency of the selected nucleic acid regions from the first and second chromosomes of interest, comparing the relative frequency of the selected nucleic acid regions from the first and second chromosomes of interest, and identifying the presence or absence of a fetal aneuploidy based on the compared relative frequencies of the selected nucleic acid regions.

In still another specific aspect, the invention provides an assay system for detection of the presence or absence of a fetal aneuploidy comprising the steps of providing a maternal sample comprising maternal and fetal cell free DNA, selectively amplifying two or more nucleic acid regions from a chromosome of interest in the maternal sample, selectively amplifying two or more nucleic acid regions from a reference chromosome in the maternal sample, determining the relative frequency of the selected nucleic acid regions from the chromosomes of interest and the reference chromosome, comparing the relative frequency of the selected nucleic acid regions from the chromosomes of interest and the reference chromosome, and identifying the presence or absence of a fetal aneuploidy based on the compared relative frequencies of the selected nucleic acid regions.

In a specific aspect, the nucleic acid regions of the first and second chromosomes are selectively amplified in a single reaction, and preferably in a single reaction contained within a single vessel. In another specific aspect, the selected nucleic acids are enriched by hybridization techniques (e.g., capture hybridization or hybridization to an array), optionally followed by one or more rounds of amplification. Optionally, the captured nucleic acids are released (e.g., by denaturation) prior to amplification and sequence determination.

The nucleic acids can be isolated from a maternal sample using various methods that allow for selective enrichment of the nucleic acids used in analysis. The isolation may be a removal of DNA in the maternal sample not used in analysis and/or removal of any excess oligonucleotides used in the initial enrichment or amplification step. For example, nucleic acids can be isolated from the maternal sample using hybridization techniques, e.g., capture using binding of the nucleic acids to complementary oligos on a solid substrate such as a bead or an array, followed by removal of the non-bound nucleic acids from the sample. In another example, when a padlock probe technique is used for selective amplification, the circularized nucleic acid products can be isolated from the linear nucleic acids, which are subject to selective degradation. In yet another example, the primers used for selective amplification may comprise a binding agent (e.g., biotin), and the amplified nucleic acids can be isolated from the remainder of the starting materials via selective binding to a partner of the binding agent (e.g., binding to avidin or streptavidin) on a solid substrate. Other useful methods of isolation will be apparent to one skilled in the art upon reading the present specification.

Preferably, the selected nucleic acids are amplified using universal amplification methods following the initial selective amplification or enrichment from the mixed sample. The use of universal amplification allows multiple nucleic acids regions to be amplified using a single or limited number of amplification primers, and is especially useful in amplifying multiple selected nucleic acid regions in a single reaction. This allows the simultaneous processing of multiple nucleic acid regions from a single or multiple samples.

Thus, in a preferred aspect of the invention, sequences complementary to primers for use in universal amplification (e.g., nucleic acid adapters) are introduced to the selected nucleic acid regions during or following selective amplification or enrichment. Preferably such sequences are introduced to the ends of selected nucleic acids, although they may be introduced in any location that allows identification of the amplification product from the universal amplification procedure.

Preferably, the assay system detects the presence or absence of genetic abnormalities in samples that can be easily obtained from a subject, such as blood, plasma, serum and the like. In one general aspect, the assay system utilizes detection of selected nucleic acid regions in cell free DNA in a mixed sample to identify the presence or absence of a copy number variation in a genomic region of interest. In one more specific aspect, the assay system utilizes detection of selected nucleic acid regions in cell free DNA in a mixed sample to identify the presence or absence of a chromosomal aneuploidy. The quantities of selected nucleic acid regions can be determined for a genomic region of interest and compared to the quantities of selected nucleic acid regions from another genomic region of interest and/or to the quantities of selected nucleic acid regions from a reference genomic region of interest to detect potential aneuploidies based on chromosome frequencies in the mixed sample.

In a particular aspect, the ratio of the frequencies of the nucleic acid are compared to a reference mean ratio that has been determined for a statistically significant population of genetically “normal” subjects, i.e. subjects that do not have the particular genetic anomaly that is being interrogated in a particular assay system.

It is a feature of the present invention that the nucleic acid regions are determined using non-polymorphic detection methods, i.e., detection methods that are not dependent upon the presence or absence of a particular polymorphism to identify the selected nucleic acid region. In a preferred aspect, the assay detection systems utilize non-polymorphic detection methods to “count” the relative numbers of selected nucleic acid regions present in a mixed sample. These numbers can be utilized to determine if, statistically, a mixed sample is likely to have a copy number variation in a genomic region. Such information can be used to identify a particular pathology or genetic disorder, to confirm a diagnosis or recurrence of a disease or disorder, to determine the prognosis of a disease or disorder, and/or to assist in determining potential treatment options.

In some aspects, the relative frequencies of selected nucleic acid regions from different chromosomes in a sample are individually quantified and compared to determine the presence or absence of an aneuploidy in a mixed sample. The individually quantified regions may undergo a normalization calculation or the data may be subjected to outlier exclusion prior to comparison to determine the presence or absence of an aneuploidy in a mixed sample. In other aspects, the relative frequencies of the selected nucleic acid regions are used to determine a chromosome frequency of the first and second chromosomes of interest, and the presence or absence of an aneuploidy is based on the compared chromosome frequencies of the first and second chromosomes of interest. In yet other aspects, the relative frequencies of the selected nucleic acid regions are used to determine a chromosome frequency of a chromosome of interest and a reference chromosome, and the presence or absence of an aneuploidy is based on the compared chromosome frequencies of the chromosome of interest and the reference chromosome.

The assay system of the invention can be configured as a highly multiplexed system which allows for multiple nucleic acid regions from a single or multiple chromosomes within an individual sample and/or multiple samples to be analyzed simultaneously. In such multiplexed systems, the samples can be analyzed separately, or they may be initially pooled into groups of two or more for analysis of larger numbers of samples. When pooled data is obtained, such data is preferably identified for the different samples prior to analysis of aneuploidy. In some aspects, however, the pooled data may be analyzed for potential aneuploidies, and individual samples from the group subsequently analyzed if initial results indicate that a potential aneuploidy is detected within the pooled group.

In certain aspects, the assay systems utilize one or more indices that provide information for sample or locus identification. For example, a primer that is used in selective amplification may have additional sequences that are specific to a locus, e.g., a nucleic acid tag sequence that is indicative of the selected nucleic acid region or a particular allele of that nucleic acid region. In another example, an index is used in selective or universal amplification that is indicative of a sample from which the nucleic acid was amplified. In yet another example, a unique identification index is used to distinguish a particular amplification product from other amplification products obtained from the detection methods. A single index may also be combined with any other index to create one index that provides information for two properties (e.g., sample-identification index, allele-locus index).

In one particular aspect, the method of the invention generally comprises detection of the number of copies of two or more selected nucleic acid regions corresponding to a first chromosome and two or more selected nucleic acid regions corresponding to a second chromosome, and comparison of the quantities of the selected nucleic acids in a maternal sample to identify the presence or absence of fetal aneuploidy. The selected nucleic acid regions can be isolated from the maternal sample using any means that selectively isolate the particular nucleic acids present in the maternal sample for analysis, e.g., hybridization, selective amplification or other form of sequence-based isolation of the nucleic acids from the maternal sample. Following isolation, the selected target nucleic acids are individually distributed in a suitable detection format, e.g., on a microarray or in a flow cell, for determination of the relative quantities of each selected nucleic acid in the maternal sample. The relative quantities of the detected nucleic acids are indicative of the number of copies of chromosomes that correspond to the target nucleic acids present in the maternal sample.

Following isolation and distribution of the target nucleic acids in a suitable format, the target sequences are identified, e.g., through sequence determination of the target sequence itself or via detection of an associated index (e.g., an identification index, a locus index, an allele index and the like).

In one specific aspect, the invention provides an assay system for detection of the presence or absence of a fetal aneuploidy, comprising the steps of providing a maternal sample comprising maternal and fetal cell free DNA, amplifying two or more selected nucleic acid regions from a first chromosome of interest in the maternal sample, amplifying two or more selected nucleic acid regions from a second chromosome of interest in the maternal sample, determining the relative frequency of the selected regions from the chromosomes of interest, comparing the relative frequency of the selected nucleic acid regions from the first and second chromosomes of interest, and identifying the presence or absence of a fetal aneuploidy based on the compared relative frequencies of the selected nucleic acid regions.

In some specific aspects, the relative frequencies of the nucleic acid regions are individually calculated, and the relative frequencies of the individual nucleic acid regions are compared to determine the presence or absence of a fetal aneuploidy. In other specific aspects, the relative frequencies of the selected regions are used to determine a chromosome frequency of the first and second chromosomes of interest, and the presence or absence of a fetal aneuploidy is based on the compared chromosome frequencies of the first and second chromosomes of interest.

In another specific aspect, the invention provides an assay system for detection of the presence or absence of a fetal aneuploidy, comprising the steps of providing a maternal sample comprising maternal and fetal cell free DNA, amplifying two or more selected nucleic acid regions from a chromosome of interest in the maternal sample, amplifying two or more selected nucleic acid regions from a reference chromosome in the maternal sample, determining the relative frequency of the selected regions from the chromosomes of interest and the reference chromosome, comparing the relative frequency of the selected nucleic acid regions from the chromosomes of interest and the reference chromosome, and identifying the presence or absence of a fetal aneuploidy based on the compared relative frequencies of the selected nucleic acid regions. In some specific aspects, the relative frequencies of the nucleic acid regions are individually calculated, and the relative frequencies of the individual nucleic acid regions are compared to determine the presence or absence of a fetal aneuploidy. In other specific aspects, the relative frequencies of the nucleic acid regions are used to determine a chromosome frequency of the chromosome of interest and the reference chromosome, and the presence or absence of a fetal aneuploidy is based on the compared chromosome frequencies of the chromosome of interest and the reference chromosome.

The maternal sample used for analysis can be obtained or derived from any sample which contains the nucleic acid of interest to be analyzed using the assay system of the invention. For example, a maternal sample may be from any maternal fluid which comprises both maternal and fetal cell free DNA, including but not limited to maternal plasma, maternal serum, or maternal blood.

It is a feature of the invention that the nucleic acids analyzed in the assay system do not require polymorphic differences between the fetal and maternal sequences to determine potential aneuploidy. It is another feature of the invention that the substantial majority of the nucleic acids isolated from the maternal sample and detected in the assay system provide information relevant to the presence and quantity of a particular chromosome in the maternal sample, i.e. the detected target nucleic acids are indicative of a particular nucleic acid region associated with a chromosome. This ensures that the majority of nucleic acids analyzed in the assay system of the invention can be used in analysis, differentiating it from techniques such as MPSS which only utilize a subset of the generated sequence data.

In some aspects, multiple nucleic acid regions are determined for each genomic region under interrogation, and the quantity of the selected regions present in the maternal sample are individually summed to determine the relative frequency of a nucleic acid region in a maternal sample. This includes determination of the frequency of the nucleic acid region for the combined maternal and fetal DNA present in the maternal sample. Preferably, the determination does not require a distinction between the maternal and fetal DNA, although in certain aspects this information may be obtained in addition to the information of relative frequencies in the sample as a whole.

In preferred aspects, target nucleic acids corresponding to multiple nucleic acid regions from a chromosome are detected and summed to determine the relative frequency of a chromosome in the maternal sample. Frequencies that are higher than expected for a nucleic acid region corresponding to one chromosome when compared to the quantity of a nucleic acid region corresponding to another chromosome in the maternal sample are indicative of a fetal duplication, deletion or aneuploidy. The comparison can be comparison of a genomic region of interest that is putatively inserted or deleted in a fetal chromosome. The comparison can also be of chromosomes that each may be a putative aneuploid in the fetus (e.g., chromosomes 18 and 21), where the likelihood of both being aneuploid is minimal. The comparison can also be of chromosomes where one is putatively aneuploid (e.g., chromosome 21) and the other acts as a reference chromosome (e.g., an autosome such as chromosome 12). In yet other aspects, the comparison may utilize two or more chromosomes that are putatively aneuploid and one or more reference chromosomes.

In one aspect, the assay system of the invention analyzes multiple nucleic acids representing selected loci on chromosomes of interest, and the relative frequency of each selected locus from the sample is analyzed to determine a relative chromosome frequency for each particular chromosome of interest in the sample. The chromosomal frequency of two or more chromosomes is then compared to statistically determine whether a chromosomal abnormality exists.

In another aspect, the assay system of the invention analyzes multiple nucleic acids representing selected loci on chromosomes of interest, and the relative frequency of each selected nucleic acid from the sample is analyzed and independently quantified to determine a relative amount for each selected locus in the sample. The sum of the loci in the sample is compared to statistically determine whether a chromosomal aneuploidy exists.

In another aspect, subsets of loci on each chromosome are analyzed to determine whether a chromosomal abnormality exists. The loci frequency can be summed for a particular chromosome, and the summations of the loci used to determine a duplication, deletion or an aneuploidy. This aspect of the invention sums the frequencies of the individual loci on each chromosome and then compares the sum of the loci on one chromosome against another chromosome to determine whether a chromosomal abnormality exists. The subsets of loci can be chosen randomly but with sufficient numbers of loci to yield a statistically significant result in determining whether a chromosomal abnormality exists. Multiple analyses of different subsets of loci can be performed within a mixed sample to yield more statistical power. In another aspect, particular loci can be selected on each chromosome that are known to have less variation between maternal samples, or by limiting the data used for determination of chromosomal frequency, e.g., by ignoring the data from loci with very high or very low frequency within a sample.

In a particular aspect, the measured quantity of one or more selected loci on a chromosome is normalized to account for differences in loci quantity in the sample. This can be done by normalizing for known variation from sources such as the assay system (e.g., temperature, reagent lot differences), underlying biology of the sample (e.g., nucleic acid content), operator differences, or any other variables.

In certain specific aspects, determining the relative percentage of fetal DNA in a maternal sample may be beneficial in performing the assay system, as it will provide important information on the relative statistical presence of nucleic acid regions that may be indicative of fetal aneuploidy. In each maternally-derived sample, the fetus will have approximately 50% of its loci inherited from the mother and 50% of the loci inherited from the father when no copy number variant is present for that locus. Determining the loci contributed to the fetus from non-maternal sources (e.g., through identification of Y-specific sequences, polymorphisms, or de novo fetal mutations) can allow the estimation of fetal DNA in a maternal sample, and thus provide information used to calculate the statistically significant differences in chromosomal frequencies for chromosomes of interest. Such loci could thus provide two forms of information in the assay—allelic information can be used for determining the percent fetal DNA contribution in a maternal sample and a summation of the allelic information can be used to determine the relative overall frequency of that locus in a maternal sample. The allelic information is not needed to determine the relative overall frequency of that locus.

Thus, in some specific aspects, the relative fetal contribution of maternal DNA at the allele of interest can be compared to the non-maternal contribution at that allele to determine approximate fetal DNA concentration in the sample. In a particular aspect, the estimation of fetal DNA in a maternal sample is determined at those loci where the mother is homozygous at the locus for a given allele and a different allele is present in the fetus, e.g., inherited from the father at that locus or which possesses a de novo fetal mutation. In this situation, the fetal DNA amount will be approximately twice the amount of the fetal allele inherited from the father. In other specific aspects, the relative quantity of solely paternally-derived sequences (e.g., Y-chromosome sequences or paternally-specific polymorphisms) can be used to determine the relative concentration of fetal DNA in a maternal sample.

In certain specific aspects, determining the relative percentage of fetal DNA in a maternal sample may be beneficial in performing or optimizing results obtained from the assay system, as it will provide important information on the expected statistical presence of the fetal chromosomes and deviation from that expectation may be indicative of fetal aneuploidy. Numerous approaches can be used to calculate the relative contribution of fetal DNA in a maternal sample.

In some aspects, the percent fetal DNA contribution in a maternal sample is calculated by detecting levels of one or more non-maternally contributed loci, including loci on the Y-chromosome and autosomal loci. In aspects utilizing autosomal loci, generally the percent fetal DNA contribution is determined by comparing one or more genetic variations on the non-maternal loci to the maternal loci. In some particular aspects, these genetic variations are copy number variations. In other particular aspects, these genetic variations are one or more single nucleotide polymorphisms.

In other aspects, the percent fetal DNA contribution in a maternal sample is calculated by detecting methylation differences between loci on fetal DNA and maternal DNA.

The amplified molecules in the assay samples are analyzed to determine a first number of assay samples which contain the selected genetic sequence and a second number of assay samples which contain a reference genetic sequence.

Thus, in a specific aspect, the invention provides an assay system for detection of the presence or absence of a fetal aneuploidy comprising the steps of providing a maternal sample comprising maternal and fetal cell free DNA, amplifying two or more selected polymorphic nucleic acid regions from a chromosome, detecting the amplified nucleic acid regions, quantifying the relative frequency of each allele from the selected polymorphic nucleic acid regions to determine the percent fetal cell free DNA in the maternal sample, selectively amplifying two or more selected nucleic acid regions from a first chromosome of interest in the maternal sample, selectively amplifying two or more selected nucleic acid regions from a second chromosome of interest in the maternal sample, detecting the amplified nucleic acid regions, quantifying the relative frequency of the selected nucleic acid regions from the first and second chromosomes of interest, comparing the relative frequency of the selected nucleic acid regions from the first and second chromosomes of interest in view of the percent fetal cell free DNA in the sample, and determining the presence or absence of a chromosomal aneuploidy based on the relative frequency. The assay system can use the percent fetal cell free DNA in the sample to optimize the statistical likelihood of the presence or absence of a fetal aneuploidy by adjusting the relative frequency based on the comparison of the selected nucleic acid regions from the first and second chromosomes of interest.

In some aspects, the relative frequencies of each nucleic acid region for each chromosome are summed and the sums for each chromosome compared to calculate a chromosomal ratio. In specific aspects, the chromosomal ratio is compared to the mean chromosomal ratio from a normal population and the threshold for identifying the presence or absence of an aneuploidy is at least three times the chromosomal variation in the normal population.

In certain aspects, the percent fetal contribution is calculated by detecting levels of one or more non-maternally contributed loci. These loci may be, e.g., loci on the Y-chromosome or autosomal loci that differ between the fetus and the mother. When the non-maternal loci are autosomal, they preferably comprise one or more genetic variations compared to the maternal loci, such as a copy number variation or a single nucleotide polymorphisms. In other aspects, the percent fetal contribution is calculating using methylation differences between fetal DNA and maternal DNA.

In another specific aspect, the invention provides an assay system for determination of the percent fetal cell free DNA concentration in a maternal sample comprising the steps of providing a maternal sample comprising maternal and fetal cell free DNA, amplifying two or more selected polymorphic nucleic acid regions from a normal, autosomal chromosome, detecting the amplified nucleic acid regions, quantifying the relative frequency of each allele from the selected polymorphic nucleic acid regions, selecting polymorphic nucleic acid regions where the maternal DNA is homozygous and the fetal DNA is heterozygous, computing a sum of the low frequency alleles for such polymorphic nucleic acid regions, computing a sum of the high frequency alleles for such polymorphic nucleic acid regions and dividing the sum of the low frequency allele by the sum of the high and low frequency alleles and multiplying by two to calculate the percent contribution of fetal cell free DNA in the maternal sample.

The invention also provides an assay system for determination of the percent fetal cell free DNA concentration in a maternal sample comprising the steps of providing a maternal sample comprising maternal and fetal cell free DNA, amplifying two or more selected polymorphic nucleic acid regions from an autosomal chromosome, detecting the amplified nucleic acid regions, quantifying the relative frequency of each allele from the selected polymorphic nucleic acid regions, selecting polymorphic nucleic acid regions where the maternal DNA is homozygous and the fetal DNA is heterozygous, computing an average of the low frequency alleles for such polymorphic nucleic acid regions, computing an average of the high frequency alleles for such polymorphic nucleic acid regions; dividing the average of the low frequency alleles by the average of the high and low frequency alleles and multiplying by two to calculate the percent contribution of fetal cell free DNA in the maternal sample. The information on percent fetal cell free DNA can be used in conjunction with certain assay systems of the invention used to determine the statistical likelihood of the presence or absence of a fetal aneuploidy.

The assay system preferably analyzes at least twenty-four polymorphic nucleic acid regions for each chromosome of interest, more preferably at least forty-eight polymorphic nucleic acid regions for each chromosome of interest, and even more preferably at least ninety-six polymorphic nucleic acid regions for each chromosome of interest.

In a specific aspect, the assay system of the invention can be utilized to determine if one or more fetus in a multiples pregnancy is likely to have an aneuploidy, and whether further confirmatory tests should be undertaken to confirm the identification of the fetus with the abnormality. For example, the assay system of the invention can be used to determine if one of two twins has a high likelihood of an aneuploidy, followed by a more invasive technique that can distinguish physically between the fetuses, such as amniocentesis or chorionic villi sampling, to determine the identification of the affected fetus.

In another specific aspect, the assay system of the invention can be utilized to determine if a fetus has a potential mosaicism, and whether further confirmatory tests should be undertaken to confirm the identification of mosaicism in the fetus. Mosaicism could be subsequently confirmed using other testing methods that could distinguish mosaic aneuploidy in specific cells or tissue, either prenatally or postnatally.

In another aspect, the oligonucleotides used for isolation of a selected nucleic acid can be connected at the non-sequence specific ends such that a circular or unimolecular probe may be formed. In this aspect, the 3′ end and the 5′ end of the circular probe binds to the target sequence and at least one universal amplification region is present in the non-target specific sequence of the circular probe.

In certain aspects, the assay format allows the detection of a combination of abnormalities using different detection mechanisms applied to the maternal sample. For example, fetal aneuploidy can be determined through the identification of selected target nucleic acids in a maternal sample, and specific mutations may be detected by sequence determination of mutations in one or more identified alleles of a known locus. Thus, in specific aspects, sequence determination of a target nucleic acid can provide information on the number of copies of a particular locus in a maternal sample as well as the presence of a mutation in a fetal allele within the maternal sample.

These and other aspects, features and advantages will be provided in more detail as described herein.

### BRIEF DESCRIPTION OF THE FIGURES

FIGS. 1A and 1B are graphs illustrating the Z Statistics for a first cohort of samples.

FIGS. 2A and 2B are graphs illustrating the odds scores of trisomy versus disomy for T21 and T18 from a first cohort.

FIGS. 3A and 3B are graphs illustrating the odds scores of trisomy versus disomy for T21 and T18 from a blinded cohort.

FIG. 4 is a graph illustrating the ability of the assay system to determine percent fetal DNA in a maternal sample.

### DETAILED DESCRIPTION OF THE INVENTION

The methods described herein may employ, unless otherwise indicated, conventional techniques and descriptions of molecular biology (including recombinant techniques), cell biology, biochemistry, and microarray and sequencing technology, which are within the skill of those who practice in the art. Such conventional techniques include polymer array synthesis, hybridization and ligation of oligonucleotides, sequencing of oligonucleotides, and detection of hybridization using a label. Specific illustrations of suitable techniques can be had by reference to the examples herein. However, equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, et al., Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Stryer, L., Biochemistry (4th Ed.) W.H. Freeman, New York (1995); Gait, “Oligonucleotide Synthesis: A Practical Approach” IRL Press, London (1984); Nelson and Cox, Lehninger, Principles of Biochemistry, 3^{rd} Ed., W. H. Freeman Pub., New York (2000); and Berg et al., Biochemistry, 5^{th} Ed., W.H. Freeman Pub., New York (2002), all of which are herein incorporated by reference in their entirety for all purposes. Before the present compositions, research tools and methods are described, it is to be understood that this invention is not limited to the specific methods, compositions, targets and uses described, as such may, of course, vary. It is also to be understood that the terminology used herein is for the purpose of describing particular aspects only and is not intended to limit the scope of the present invention, which will be limited only by appended claims.

It should be noted that as used herein and in the appended claims, the singular forms “a,” “and,” and “the” include plural referents unless the context clearly dictates otherwise. Thus, for example, reference to “a nucleic acid region” refers to one, more than one, or mixtures of such regions, and reference to “an assay” includes reference to equivalent steps and methods known to those skilled in the art, and so forth.

Where a range of values is provided, it is to be understood that each intervening value between the upper and lower limit of that range—and any other stated or intervening value in that stated range—is encompassed within the invention. Where the stated range includes upper and lower limits, ranges excluding either of those included limits are also included in the invention.

Unless expressly stated, the terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated. All publications mentioned herein are incorporated by reference for the purpose of describing and disclosing the formulations and methodologies that are described in the publication and which might be used in connection with the presently described invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

### DEFINITIONS

The terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

The term “amplified nucleic acid” is any nucleic acid molecule whose amount has been increased at least two fold by any nucleic acid amplification or replication method performed in vitro as compared to its starting amount.

The term “chromosomal abnormality” refers to any genetic variant for all or part of a chromosome. The genetic variants may include but not be limited to any copy number variant such as duplications or deletions, translocations, inversions, and mutations.

The terms “complementary” or “complementarity” are used in reference to nucleic acid molecules (i.e., a sequence of nucleotides) that are related by base-pairing rules. Complementary nucleotides are, generally, A and T (or A and U), or C and G. Two single stranded RNA or DNA molecules are said to be substantially complementary when the nucleotides of one strand, optimally aligned and with appropriate nucleotide insertions or deletions, pair with at least about 90% to about 95% complementarity, and more preferably from about 98% to about 100% complementarity, and even more preferably with 100% complementarity. Alternatively, substantial complementarity exists when an RNA or DNA strand will hybridize under selective hybridization conditions to its complement. Selective hybridization conditions include, but are not limited to, stringent hybridization conditions. Stringent hybridization conditions will typically include salt concentrations of less than about 1 M, more usually less than about 500 mM and preferably less than about 200 mM. Hybridization temperatures are generally at least about 2° C. to about 6° C. lower than melting temperatures (Tₘ).

The term “correction index” refers to an index that may contain additional nucleotides that allow for identification and correction of amplification, sequencing or other experimental errors including the detection of deletion, substitution, or insertion of one or more bases during sequencing as well as nucleotide changes that may occur outside of sequencing such as oligo synthesis, amplification, and any other aspect of the assay. These correction indices may be stand-alone indices that are separate sequences, or they may be embedded within other indices to assist in confirming accuracy of the experimental techniques used, e.g., a correction index may be a subset of sequences of a locus index or an identification index.

The term “diagnostic tool” as used herein refers to any composition or assay of the invention used in combination as, for example, in a system in order to carry out a diagnostic test or assay on a patient sample.

The term “hybridization” generally means the reaction by which the pairing of complementary strands of nucleic acid occurs. DNA is usually double-stranded, and when the strands are separated they will re-hybridize under the appropriate conditions. Hybrids can form between DNA-DNA, DNA-RNA or RNA-RNA. They can form between a short strand and a long strand containing a region complementary to the short one. Imperfect hybrids can also form, but the more imperfect they are, the less stable they will be (and the less likely to form).

The term “identification index” refers generally to a series of nucleotides incorporated into a primer region of an amplification process for unique identification of an amplification product of a nucleic acid region. Identification index sequences are preferably 6 or more nucleotides in length. In a preferred aspect, the identification index is long enough to have statistical probability of labeling each molecule with a target sequence uniquely. For example, if there are 3000 copies of a particular target sequence, there are substantially more than 3000 identification indexes such that each copy of a particular target sequence is likely to be labeled with a unique identification index. The identification index may contain additional nucleotides that allow for identification and correction of sequencing errors including the detection of deletion, substitution, or insertion of one or more bases during sequencing as well as nucleotide changes that may occur outside of sequencing such as oligo synthesis, amplification, and any other aspect of the assay. The index may be combined with any other index to create one index that provides information for two properties (e.g., sample-identification index, locus-identification index).

The term “likelihood” refers to any value achieved by directly calculating likelihood or any value that can be correlated to or otherwise indicative of a likelihood.

The terms “locus” and “loci” as used herein refer to a nucleic acid region of known location in a genome.

The term “locus index” refers generally to a series of nucleotides that correspond to a known locus on a chromosome. Generally, the locus index is long enough to label each known locus region uniquely. For instance, if the method uses 192 known locus regions corresponding to 192 individual sequences associated with the known loci, there are at least 192 unique locus indexes, each uniquely identifying a region indicative of a particular locus on a chromosome. The locus indices used in the methods of the invention may be indicative of different loci on a single chromosome as well as known loci present on different chromosomes within a sample. The locus index may contain additional nucleotides that allow for identification and correction of sequencing errors including the detection of deletion, substitution, or insertion of one or more bases during sequencing as well as nucleotide changes that may occur outside of sequencing such as oligo synthesis, amplification, and any other aspect of the assay.

The term “maternal sample” as used herein refers to any sample taken from a pregnant mammal which comprises both fetal and maternal cell free genomic material (e.g., DNA). Preferably, maternal samples for use in the invention are obtained through relatively non-invasive means, e.g., phlebotomy or other standard techniques for extracting peripheral samples from a subject.

The term “melting temperature” or Tₘ is commonly defined as the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+16.6(log 10[Na+])0.41(%[G+C])−675/n−1.0m, when a nucleic acid is in aqueous solution having cation concentrations of 0.5 M or less, the (G+C) content is between 30% and 70%, n is the number of bases, and m is the percentage of base pair mismatches (see, e.g., Sambrook J et al., Molecular Cloning, A Laboratory Manual, 3rd Ed., Cold Spring Harbor Laboratory Press (2001)). Other references include more sophisticated computations, which take structural as well as sequence characteristics into account for the calculation of Tₘ.

“Microarray” or “array” refers to a solid phase support having a surface, preferably but not exclusively a planar or substantially planar surface, which carries an array of sites containing nucleic acids such that each site of the array comprises substantially identical or identical copies of oligonucleotides or polynucleotides and is spatially defined and not overlapping with other member sites of the array; that is, the sites are spatially discrete. The array or microarray can also comprise a non-planar interrogatable structure with a surface such as a bead or a well. The oligonucleotides or polynucleotides of the array may be covalently bound to the solid support, or may be non-covalently bound. Conventional microarray technology is reviewed in, e.g., Schena, Ed., Microarrays: A Practical Approach, IRL Press, Oxford (2000). “Array analysis”, “analysis by array” or “analysis by microarray” refers to analysis, such as, e.g., isolation of specific nucleic acids or sequence analysis of one or more biological molecules using a microarray.

The term “mixed sample” as used herein refers to any sample comprising cell free genomic material (e.g., DNA) from two or more cell types of interest. Exemplary mixed samples include a maternal sample (e.g., maternal blood, serum or plasma comprising both maternal and fetal DNA), and a peripherally-derived somatic sample (e.g., blood, serum or plasma comprising different cell types, e.g., hematopoietic cells, mesenchymal cells, and circulating cells from other organ systems). Mixed samples include samples with genomic material from two different sources, which may be sources from a single individual, e.g., normal and atypical somatic cells, or cells that are from two different individuals, e.g., a sample with both maternal and fetal genomic material or a sample from a transplant patient that comprises cells from both the donor and recipient.

By “non-polymorphic”, when used with respect to detection of selected nucleic acid regions, is meant a detection of such nucleic acid region, which may contain one or more polymorphisms, but in which the detection is not reliant on detection of the specific polymorphism within the region. Thus a selected nucleic acid region may contain a polymorphism, but detection of the region using the assay system of the invention is based on occurrence of the region rather than the presence or absence of a particular polymorphism in that region.

As used herein “nucleotide” refers to a base-sugar-phosphate combination.

Nucleotides are monomeric units of a nucleic acid sequence (DNA and RNA). The term nucleotide includes ribonucleoside triphosphates ATP, UTP, CTG, GTP and deoxyribonucleoside triphosphates such as dATP, dCTP, dITP, dUTP, dGTP, dTTP, or derivatives thereof. Such derivatives include, for example, [αS]dATP, 7-deaza-dGTP and 7-deaza-dATP, and nucleotide derivatives that confer nuclease resistance on the nucleic acid molecule containing them. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP.

According to the present invention, a “nucleotide” may be unlabeled or detectably labeled by well known techniques. Fluorescent labels and their attachment to oligonucleotides are described in many reviews, including Haugland, Handbook of Fluorescent Probes and Research Chemicals, 9th Ed., Molecular Probes, Inc., Eugene Oreg. (2002); Keller and Manak, DNA Probes, 2nd Ed., Stockton Press, New York (1993); Eckstein, Ed., Oligonucleotides and Analogues: A Practical Approach, IRL Press, Oxford (1991); Wetmur, Critical Reviews in Biochemistry and Molecular Biology, 26:227-259 (1991); and the like. Other methodologies applicable to the invention are disclosed in the following sample of references: Fung et al., U.S. Pat. No. 4,757,141; Hobbs, Jr., et al., U.S. Pat. No. 5,151,507; Cruickshank, U.S. Pat. No. 5,091,519; Menchen et al., U.S. Pat. No. 5,188,934; Begot et al., U.S. Pat. No. 5,366,860; Lee et al., U.S. Pat. No. 5,847,162; Khanna et al., U.S. Pat. No. 4,318,846; Lee et al., U.S. Pat. No. 5,800,996; Lee et al., U.S. Pat. No. 5,066,580: Mathies et al., U.S. Pat. No. 5,688,648; and the like. Labeling can also be carried out with quantum dots, as disclosed in the following patents and patent publications: U.S. Pat. Nos. 6,322,901; 6,576,291; 6,423,551; 6,251,303; 6,319,426; 6,426,513; 6,444,143; 5,990,479; 6,207,392; 2002/0045045; and 2003/0017264. Detectable labels include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′ dimethylaminophenylazo) benzoic acid (DABCYL), CASCADE BLUE® (pyrenyloxytrisulfonic acid), OREGON GREEN™ (2′,7′-difluorofluorescein), TEXAS RED™ (sulforhodamine 101 acid chloride), Cyanine and 5-(2′-aminoethyl)aminonaphthalene-1-sulfonic acid (EDANS). Specific examples of fluorescently labeled nucleotides include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif. FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, CASCADE BLUE®-7-UTP (pyrenyloxytrisulfonic acid-7-UTP), CASCADE BLUE®-7-dUTP (pyrenyloxytrisulfonic acid-7-dUTP), fluorescein-12-UTP, fluorescein-12-dUTP, OREGON GREEN™ 488-5-dUTP (2′,7′-difluorofluorescein-5-dUTP), RHODAMINE GREEN™-5-UTP ((5-{2-[4-(aminomethyl)phenyl]-5-(pyridin-4-yl)-1H-1-5-UTP)), RHODAMINE GREEN™-5-dUTP ((5-{2-[4-(aminomethyl)phenyl]-5-(pyridin-4-yl)-1H-1-5-dUTP)), tetramethylrhodamine-6-UTP, tetramethylrhodamine-6-dUTP, TEXAS RED™-5-UTP (sulforhodamine 101 acid chloride-5-UTP), TEXAS RED™-5-dUTP (sulforhodamine 101 acid chloride-5-dUTP), and TEXAS RED™-12-dUTP (sulforhodamine 101 acid chloride-12-dUTP) available from Molecular Probes, Eugene, Oreg. The terms “oligonucleotides” or “oligos” as used herein refer to linear oligomers of natural or modified nucleic acid monomers, including deoxyribonucleotides, ribonucleotides, anomeric forms thereof, peptide nucleic acid monomers (PNAs), locked nucleotide acid monomers (LNA), and the like, or a combination thereof, capable of specifically binding to a single-stranded polynucleotide by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g., 8-12, to several tens of monomeric units, e.g., 100-200 or more. Suitable nucleic acid molecules may be prepared by the phosphoramidite method described by Beaucage and Carruthers (Tetrahedron Lett., 22:1859-1862 (1981)), or by the triester method according to Matteucci, et al. (J. Am. Chem. Soc., 103:3185 (1981)), both incorporated herein by reference, or by other chemical methods such as using a commercial automated oligonucleotide synthesizer.

As used herein the term “polymerase” refers to an enzyme that links individual nucleotides together into a long strand, using another strand as a template. There are two general types of polymerase—DNA polymerases, which synthesize DNA, and RNA polymerases, which synthesize RNA. Within these two classes, there are numerous sub-types of polymerases, depending on what type of nucleic acid can function as template and what type of nucleic acid is formed.

As used herein “polymerase chain reaction” or “PCR” refers to a technique for replicating a specific piece of target DNA in vitro, even in the presence of excess non-specific DNA. Primers are added to the target DNA, where the primers initiate the copying of the target DNA using nucleotides and, typically, Taq polymerase or the like. By cycling the temperature, the target DNA is repetitively denatured and copied. A single copy of the target DNA, even if mixed in with other, random DNA, can be amplified to obtain billions of replicates. The polymerase chain reaction can be used to detect and measure very small amounts of DNA and to create customized pieces of DNA. In some instances, linear amplification methods may be used as an alternative to PCR.

The term “polymorphism” as used herein refers to any genetic changes in a loci that may be indicative of that particular loci, including but not limited to single nucleotide polymorphisms (SNPs), methylation differences, short tandem repeats (STRs), and the like.

Generally, a “primer” is an oligonucleotide used to, e.g., prime DNA extension, ligation and/or synthesis, such as in the synthesis step of the polymerase chain reaction or in the primer extension techniques used in certain sequencing reactions. A primer may also be used in hybridization techniques as a means to provide complementarity of a nucleic acid region to a capture oligonucleoitide for detection of a specific nucleic acid region.

The term “research tool” as used herein refers to any composition or assay of the invention used for scientific enquiry, academic or commercial in nature, including the development of pharmaceutical and/or biological therapeutics. The research tools of the invention are not intended to be therapeutic or to be subject to regulatory approval; rather, the research tools of the invention are intended to facilitate research and aid in such development activities, including any activities performed with the intention to produce information to support a regulatory submission.

The term “sample index” refers generally to a series of unique nucleotides (i.e., each sample index is unique to a sample in a multiplexed assay system for analysis of multiple samples). The sample index can thus be used to assist in nucleic acid region identification for multiplexing of different samples in a single reaction vessel, such that each sample can be identified based on its sample index. In a preferred aspect, there is a unique sample index for each sample in a set of samples, and the samples are pooled during sequencing. For example, if twelve samples are pooled into a single sequencing reaction, there are at least twelve unique sample indexes such that each sample is labeled uniquely. The index may be combined with any other index to create one index that provides information for two properties (e.g., sample-identification index, sample-locus index).

The term “selected nucleic acid region” as used herein refers to a nucleic acid region corresponding to an individual chromosome. Such selected nucleic acid regions may be directly isolated and enriched from the sample for detection, e.g., based on hybridization and/or other sequence-based techniques, or they may be amplified using the sample as a template prior to detection of the sequence. Nucleic acids regions for use in the assay systems of the present invention may be selected on the basis of DNA level variation between individuals, based upon specificity for a particular chromosome, based on CG content and/or required amplification conditions of the selected nucleic acid regions, or other characteristics that will be apparent to one skilled in the art upon reading the present disclosure.

The term “selective amplification”, “selectively amplify” and the like refers to an amplification procedure that depends in whole or in part on hybridization of an oligo to a sequence in a selected genomic region. In certain selective amplifications, the primers used for amplification are complementary to a selected genomic region. In other selective amplifications, the primers used for amplification are universal primers, but they only result in a product if a region of the nucleic acid used for amplification is complementary to a genomic region of interest.

The terms “sequencing”, “sequence determination” and the like as used herein refers generally to any and all biochemical methods that may be used to determine the order of nucleotide bases in a nucleic acid.

The term “specifically binds”, “specific binding” and the like as used herein, when referring to a binding partner (e.g., a nucleic acid probe or primer, antibody, etc.) that results in the generation of a statistically significant positive signal under the designated assay conditions. Typically the interaction will subsequently result in a detectable signal that is at least twice the standard deviation of any signal generated as a result of undesired interactions (background).

The term “universal” when used to describe an amplification procedure refers to the use of a single primer or set of primers for a plurality of amplification reactions. For example, in the detection of 96 different target sequences, all the templates may share the identical universal priming sequences, allowing for the multiplex amplification of the 96 different sequences using a single set of primers. The use of such primers greatly simplifies multiplexing in that only two primers are needed to amplify a plurality of selected nucleic acid sequences. The term “universal” when used to describe a priming site is a site to which a universal primer will hybridize. In general,

It should also be noted that “sets” of universal priming sequences/primers may be used. For example, in highly multiplexed reactions, it may be useful to use several sets of universal sequences, rather than a single set; for example, 96 different nucleic acids may have a first set of universal priming sequences, and the second 96 a different set of universal priming sequences, etc.

### THE INVENTION IN GENERAL

The present invention provides improved methods for identifying copy number variants of particular genomic regions, including complete chromosomes (e.g., aneuploidies), in mixed samples. The detection methods of the invention are not reliant upon the presence or absence of any polymorphic or mutation information, and thus are conceptually agnostic as to the genetic variation that may be present in any chromosomal region under interrogation. These methods are useful for any mixed sample containing cell free genomic material (e.g., DNA) from two or more cell types of interest, e.g., mixed samples comprising maternal and fetal cell free DNA, mixed samples comprising cell free DNA from normal and putatively malignant cells, mixed samples comprising cell free DNA from a transplant donor and recipient, and the like.

The assay methods of the invention provide a selected enrichment of nucleic acid regions from chromosomes of interest and/or reference chromosomes for copy number variant detection. A distinct advantage of the invention is that the selected nucleic acid regions can be further analyzed using a variety of detection and quantification techniques, including but not limited to hybridization techniques, digital PCR and high throughput sequencing determination techniques. Selection probes can be designed against any number of nucleic acid regions for any chromosome. Although amplification prior to the identification and quantification of the selection nucleic acids regions is not mandatory, limited amplification prior to detection is preferred.

The present invention provides an improved system over more random techniques such as massively parallel sequencing, shotgun sequencing, and the use of random digital PCR which have been used by others to detect copy number variations in mixed samples such as maternal blood. These aforementioned approaches rely upon sequencing of all or a statistically significant population of DNA fragments in a sample, followed by mapping of these fragments or otherwise associating the fragments to their appropriate chromosomes. The identified fragments are then compared against each other or against some other reference (e.g., normal chromosomal makeup) to determine copy number variation of particular chromosomes. These methods are inherently inefficient from the present invention, as the primary chromosomes of interest only constitute a minority of data that is generated from the detection of such DNA fragments in the mixed samples.

Techniques that are dependent upon a very broad sampling of DNA in a sample provide a broad coverage of the DNA analyzed, but in fact are sampling the DNA contained within a sample on a 1× or less basis (i.e., subsampling). In contrast, the selective amplification and/or enrichment used in the present assays are specifically designed to provide depth of coverage of particular nucleic acids of interest, and provide a “super-sampling” of such selected regions with an average sequence coverage of preferably 2× or more, more preferably sequence coverage of 100× of more, even more preferably sequence coverage of 1000× or more of the selected nucleic acids present in the initial mixed sample.

The methods of the invention thus provide a more efficient and economical use of data, and the substantial majority of sequences analyzed following sample amplification result in affirmative information about the presence of a particular chromosome in the sample. Thus, unlike techniques relying on massively parallel sequencing or random digital “counting” of chromosome regions and subsequent identification of relevant data from such counts, the assay system of the invention provides a much more efficient use of data collection than the random approaches taught by others in the art.

The substantial majority of sequences analyzed are informative of the presence of a region on a chromosome of interest and/or a reference chromosome. These techniques do not require the analysis of large numbers of sequences which are not from the chromosomes of interest and which do not provide information on the relative quantity of the chromosomes of interest.

### Detecting Chromosomal Aneuploidies

The present invention provides methods for identifying fetal chromosomal aneuploidies in maternal samples comprising both maternal and fetal DNA. This can be performed using enrichment and/or amplification methods for identification of nucleic acid regions corresponding to specific chromosomes of interest and/or reference chromosomes in the maternal sample.

The assay systems utilize nucleic acids designed to enrich, isolate and/or amplify selected nucleic acids regions in a mixed sample that correspond to individual chromosomes of interest and, in certain aspects, to reference chromosomes that are used to determine the presence or absence of aneuploidy in a mixed sample. These probes are specifically designed to hybridize to a selected nucleic acid region of a particular chromosome, and thus quantification of the nucleic acid regions in a mixed sample using these probes is indicative of the copy number of a particular chromosome in the mixed sample.

The assay systems of the invention preferably employ one or more selective amplification or enrichment steps (e.g., using one or more primers that specifically hybridize to a selected nucleic acid region) to enhance the DNA content of a sample and/or to provide improved mechanisms for isolating, amplifying or analyzing the selected nucleic acid regions. This is in direct contrast to the random amplification approach used by others employing, e.g., MPSS, as such amplification techniques generally involve random amplification of all or a substantial portion of the genome.

In a general aspect, the user of the invention analyzes multiple target sequences on different chromosomes and simultaneously determines the frequency or amount of the target sequences of the chromosomes. When multiple target sequences are analyzed on chromosomes, a preferred embodiment is to amplify all of the target sequences for each sample in one reaction vessel. The target sequences from multiple samples can be amplified in one reaction vessel, and the sample of origin of the different amplification products can be determined by use of an identification index. The frequency or amount of the multiple target sequences on the different chromosomes is then compared to determine whether a chromosomal abnormality exists.

The user of the invention can also analyze multiple target sequences on multiple chromosomes and average the frequency of the target sequences on the multiple chromosomes together. Normalization or standardization of the frequencies can be performed for one or more target sequences.

In some aspects, the user of the invention sums the frequencies of the target sequences on each chromosome and then compares the sum of the target sequences on one chromosome against another chromosome to determine whether a chromosomal abnormality exists. Alternatively, one can analyze subsets of target sequences on each chromosome to determine whether a chromosomal abnormality exists. The comparison can be made either within the same or different chromosomes.

The data used to determine the frequency of the target sequences may exclude outlier data that appear to be due to experimental error, or that have elevated or depressed levels based on an idiopathic genetic bias within a particular sample. In one example, the data used for summation may exclude DNA regions with a particularly elevated frequency in one or more samples. In another example, the data used for summation may exclude target sequences that are found in a particularly low abundance in one or more samples.

Subsets of loci can be chosen randomly but with sufficient numbers of loci to yield a statistically significant result in determining whether a chromosomal abnormality exists. Multiple analyses of different subsets of loci can be performed within a mixed sample to yield more statistical power. For example, if there are 100 selected regions for chromosome 21 and 100 selected regions for chromosome 18, a series of analyses could be performed that evaluate fewer than 100 regions for each of the chromosomes. In this example, target sequences are not being selectively excluded.

The quantity of different nucleic acids detectable on certain chromosomes may vary depending upon a number of factors, including general representation of fetal loci in maternal samples, degradation rates of the different nucleic acids representing fetal loci in maternal samples, sample preparation methods, and the like. Thus, in another aspect, the quantity of particular loci on a chromosome are summed to determine the loci quantity for different chromosomes in the sample. The loci frequency are summed for a particular chromosome, and the sum of the loci are used to determine aneuploidy. This aspect of the invention sums the frequencies of the individual loci on each chromosome and then compares the sum of the loci on one chromosome against another chromosome to determine whether a chromosomal abnormality exists.

The nucleic acids analyzed using the assay systems of the invention are preferably selectively amplified and optionally isolated from the mixed sample using primers specific to the nucleic acid region of interest (e.g., to a locus of interest in a maternal sample). The primers for such selective amplification designed to isolate regions may be chosen for various reasons, but are preferably designed to 1) efficiently amplify a region from the chromosome of interest; 2) have a predictable range of expression from maternal and/or fetal sources in different maternal samples; 3) be distinctive to the particular chromosome, i.e., not amplify homologous regions on other chromosomes. The following are exemplary techniques that may be employed in the assay system or the invention.

### Amplification Methods

Numerous amplification methods can be used to provide the amplified nucleic acids that are analyzed in the assay systems of the invention, and such methods are preferably used to increase the copy numbers of a nucleic acid region of interest in a mixed sample in a manner that allows preservation of information concerning the initial content of the nucleic acid region in the mixed sample. Although not all combinations of amplification and analysis are described herein in detail, it is well within the skill of those in the art to utilize different amplification methods and/or analytic tools to isolate and/or analyze the nucleic acids of region consistent with this specification, and such variations will be apparent to one skilled in the art upon reading the present disclosure.

Such amplification methods include but are not limited to, polymerase chain reaction (PCR) (U.S. Pat. Nos. 4,683,195; and 4,683,202; PCR Technology: Principles and Applications for DNA Amplification, ed. H. A. Erlich, Freeman Press, NY, N.Y., 1992), ligase chain reaction (LCR) (Wu and Wallace, Genomics 4:560, 1989; Landegren et al., Science 241:1077, 1988), strand displacement amplification (SDA) (U.S. Pat. Nos. 5,270,184; and 5,422,252), transcription-mediated amplification (TMA) (U.S. Pat. No. 5,399,491), linked linear amplification (LLA) (U.S. Pat. No. 6,027,923), and the like, self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NASBA). (See, U.S. Pat. Nos. 5,409,818, 5,554,517, and 6,063,603, each of which is incorporated herein by reference). Other amplification methods that may be used include: Qbeta Replicase, described in PCT Patent Application No. PCT/US87/00880, isothermal amplification methods such as SDA, described in Walker et al. 1992, Nucleic Acids Res. 20(7):1691-6, 1992, and rolling circle amplification, described in U.S. Pat. No. 5,648,245. Other amplification methods that may be used are described in, U.S. Pat. Nos. 5,242,794, 5,494,810, 4,988,617 and in U.S. Ser. No. 09/854,317 and US Pub. No. 20030143599, each of which is incorporated herein by reference. In some aspects DNA is amplified by multiplex locus-specific PCR. In a preferred aspect the DNA is amplified using adaptor-ligation and single primer PCR. Other available methods of amplification include balanced PCR (Makrigiorgos, et al. (2002), Nat Biotechnol, Vol. 20, pp. 936-9) and self-sustained sequence replication (Guatelli et al., Proc. Natl. Acad. Sci. USA 87: 1874, 1990). Based on such methodologies, a person skilled in the art can readily design primers in any suitable regions 5′ and 3′ to a nucleic acid region of interest. Such primers may be used to amplify DNA of any length so long that it contains the nucleic acid region of interest in its sequence.

The length of an amplified selected nucleic acid from a genomic region of interest is generally long enough to provide enough sequence information to distinguish it from other nucleic acids that are amplified and/or selected. Generally, an amplified nucleic acid is at least about 16 nucleotides in length, and more typically, an amplified nucleic acid is at least about 20 nucleotides in length. In a preferred aspect of the invention, an amplified nucleic acid is at least about 30 nucleotides in length. In a more preferred aspect of the invention, an amplified nucleic acid is at least about 32, 40, 45, 50, or 60 nucleotides in length. In other aspects of the invention, an amplified nucleic acid can be about 100, 150 or up to 200 in length.

In certain aspects, the selective amplification uses one or a few rounds of amplification with primer pairs comprising nucleic acids complementary to the selected nucleic acids. In other aspects, the selective amplification comprises an initial linear amplification step. These methods can be particularly useful if the starting amount of DNA is quite limited, e.g., where the cell-free DNA in a sample is available in limited quantities. This mechanism increases the amount of DNA molecules that are representative of the original DNA content, and help to reduce sampling error where accurate quantification of the DNA or a fraction of the DNA (e.g., fetal DNA contribution in a maternal sample) is needed.

Thus, in one aspect, a limited number of cycles of sequence-specific amplification are performed on the starting maternal sample comprising cell free DNA. The number of cycles is generally less than that used for a typical PCR amplification, e.g., 5-30 cycles or fewer. Primers or probes may be designed to amplify specific genomic segments or regions. The primers or probes may be modified with an end label at the 5′ end (e.g., with biotin) or elsewhere along the primer or probe such that the amplification products could be purified or attached to a solid substrate (e.g., bead or array) for further isolation or analysis. In a preferred aspect, the primers are multiplexed such that a single reaction yields multiple DNA fragments from different regions. Amplification products from the linear amplification could then be further amplified with standard PCR methods or with additional linear amplification.

For example, cell free DNA can be isolated from blood, plasma, or serum from a pregnant woman, and incubated with primers against a set number of nucleic acid regions that correspond to chromosomes of interest. Preferably, the number of primer pairs used for initial amplification will be 12 or more, more preferably 24 or more, more preferably 36 or more, even more preferably 48 or more, and even more preferably 96 or more. Each of the primers corresponds to a single nucleic acid region, and is optionally tagged for identification and/or isolation. A limited number of cycles, preferably 10 or fewer, are performed. The amplification products are subsequently isolated, e.g., when the primers are linked to a biotin molecule the amplification products can be isolated via binding to avidin or streptavidin on a solid substrate. The products are then subjected to further biochemical processes such as further amplification with other primers (e.g., universal primers) and/or detection techniques such as sequence determination and hybridization.

Efficiencies of amplification may vary between sites and between cycles so that in certain systems normalization may be used to ensure that the products from the amplification are representative of the nucleic acid content starting material. One practicing the assay system of the invention can utilize information from various samples to determine variation in nucleic acid levels, including variation in different nucleic acid regions in individual samples and/or between the same nucleic acid regions in different samples following the limited initial linear amplification. Such information can be used in normalization to prevent skewing of initial levels of DNA content.

### Universal Amplification

In preferred aspects of the invention, the selectively amplified nucleic acid regions are preferably amplified following selective amplification or enrichment, either prior to or during the nucleic acid region detection techniques. In another aspect of the invention, nucleic acid regions are selectively amplified during the nucleic acid region detection technique without any prior amplification. In a multiplexed assay system, this is preferably done through use of universal amplification of the various nucleic acid regions to be analyzed using the assay systems of the invention. Universal primer sequences are added to the selectively amplified nucleic acid regions, either during or following selective amplification, so that they may be further amplified in a single universal amplification reaction. For example, these universal primer sequences may be added to the nucleic acids regions during the selective amplification process, i.e., the primers for selective amplification have universal primer sequences that flank a locus. Alternatively, adapters comprising universal amplification sequences can be added to the ends of the selected nucleic acids as adapters following amplification and isolation of the selected nucleic acids from the mixed sample.

In one exemplary aspect, nucleic acids are initially amplified from a maternal sample using primers comprising a region complementary to selected regions of the chromosomes of interest and universal priming sites. The initial selective amplification is followed by a universal amplification step to increase the number of nucleic acid regions for analysis. This introduction of primer regions to the initial amplification products allows a subsequent controlled universal amplification of all or a portion of selected nucleic acids prior to or during analysis, e.g., sequence determination.

Bias and variability can be introduced during DNA amplification, such as that seen during polymerase chain reaction (PCR). In cases where an amplification reaction is multiplexed, there is the potential that loci will amplify at different rates or efficiency. Part of this may be due to the variety of primers in a multiplex reaction with some having better efficiency (i.e. hybridization) than others, or some working better in specific experimental conditions due to the base composition. Each set of primers for a given locus may behave differently based on sequence context of the primer and template DNA, buffer conditions, and other conditions. A universal DNA amplification for a multiplexed assay system will generally introduce less bias and variability.

Accordingly, in a preferred aspect, a small number (e.g., 1-10, preferably 3-5) of cycles of selective amplification or nucleic acid enrichment in a multiplexed mixture reaction are performed, followed by universal amplification using introduced universal priming sites. The number of cycles using universal primers will vary, but will preferably be at least 10 cycles, more preferably at least 5 cycles, even more preferably 20 cycles or more. By moving to universal amplification following one or a few selective amplification cycles, the bias of having certain loci amplify at greater rates than others is reduced.

Optionally, the assay system will include a step between the selective amplification and universal amplification to remove any excess nucleic acids that are not specifically amplified in the selective amplification.

The whole product or an aliquot of the product from the selective amplification may be used for the universal amplification. The same or different conditions (e.g., polymerase, buffers, and the like) may be used in the amplification steps, e.g., to ensure that bias and variability is not inadvertently introduced due to experimental conditions. In addition, variations in primer concentrations may be used to effectively limit the number of sequence specific amplification cycles.

In certain aspects, the universal primer regions of the primers or adapters used in the assay system are designed to be compatible with conventional multiplexed assay methods that utilize general priming mechanisms to analyze large numbers of nucleic acids simultaneously in one reaction in one vessel. Such “universal” priming methods allow for efficient, high volume analysis of the quantity of nucleic acid regions present in a mixed sample, and allow for comprehensive quantification of the presence of nucleic acid regions within such a mixed sample for the determination of aneuploidy.

Examples of such assay methods include, but are not limited to, multiplexing methods used to amplify and/or genotype a variety of samples simultaneously, such as those described in Oliphant et al., U.S. Pat. No. 7,582,420, which is incorporated herein by reference.

Some aspects utilize coupled reactions for multiplex detection of nucleic acid sequences where oligonucleotides from an early phase of each process contain sequences which may be used by oligonucleotides from a later phase of the process. Exemplary processes for amplifying and/or detecting nucleic acids in samples can be used, alone or in combination, including but not limited to the methods described below, each of which are incorporated by reference in their entirety for purposes of teaching various elements that can be used in the assay systems of the invention.

In certain aspects, the assay system of the invention utilizes one of the following combined selective and universal amplification techniques: (1) LDR coupled to PCR; (2) primary PCR coupled to secondary PCR coupled to LDR; and (3) primary PCR coupled to secondary PCR. Each of these aspects of the invention has particular applicability in detecting certain nucleic acid characteristics. However, each requires the use of coupled reactions for multiplex detection of nucleic acid sequence differences where oligonucleotides from an early phase of each process contain sequences which may be used by oligonucleotides from a later phase of the process.

Barany et al., U.S. Pat. Nos. 6,852,487, 6,797,470, 6,576,453, 6,534,293, 6,506,594, 6,312,892, 6,268,148, 6,054,564, 6,027,889, 5,830,711, 5,494,810, describe the use of the ligase chain reaction (LCR) assay for the detection of specific sequences of nucleotides in a variety of nucleic acid samples.

Barany et al., U.S. Pat. Nos. 7,807,431, 7,455,965, 7,429,453, 7,364,858, 7,358,048, 7,332,285, 7,320,865, 7,312,039, 7,244,831, 7,198,894, 7,166,434, 7,097,980, 7,083,917, 7,014,994, 6,949,370, 6,852,487, 6,797,470, 6,576,453, 6,534,293, 6,506,594, 6,312,892, and 6,268,148 describe the use of the ligase detection reaction with detection reaction (“LDR”) coupled with polymerase chain reaction (“PCR”) for nucleic acid detection.

Barany et al., U.S. Pat. Nos. 7,556,924 and 6,858,412, describe the use of padlock probes (also called “precircle probes” or “multi-inversion probes”) with coupled ligase detection reaction (“LDR”) and polymerase chain reaction (“PCR”) for nucleic acid detection.

Barany et al., U.S. Pat. Nos. 7,807,431, 7,709,201, and 7,198,814 describe the use of combined endonuclease cleavage and ligation reactions for the detection of nucleic acid sequences.

Willis et al., U.S. Pat. Nos. 7,700,323 and 6,858,412, describe the use of precircle probes in multiplexed nucleic acid amplification, detection and genotyping.

Ronaghi et al., U.S. Pat. No. 7,622,281 describes amplification techniques for labeling and amplifying a nucleic acid using an adapter comprising a unique primer and a barcode.

In addition to the various amplification techniques, numerous methods of sequence determination are compatible with the assay systems of the inventions. Preferably, such methods include “next generation” methods of sequencing. Exemplary methods for sequence determination include, but are not limited to, including, but not limited to, hybridization-based methods, such as disclosed in Drmanac, U.S. Pat. Nos. 6,864,052; 6,309,824; and 6,401,267; and Drmanac et al, U.S. patent publication 2005/0191656, which are incorporated by reference, sequencing by synthesis methods, e.g., Nyren et al, U.S. Pat. Nos. 7,648,824, 7,459,311 and 6,210,891; Balasubramanian, U.S. Pat. Nos. 7,232,656 and 6,833,246; Quake, U.S. Pat. No. 6,911,345; Li et al, Proc. Natl. Acad. Sci., 100: 414-419 (2003); pyrophosphate sequencing as described in Ronaghi et al., U.S. Pat. Nos. 7,648,824, 7,459,311, 6,828,100, and 6,210,891; and ligation-based sequencing determination methods, e.g., Drmanac et al., U.S. Pat. Appln No. 20100105052, and Church et al, U.S. Pat. Appln Nos. 20070207482 and 20090018024.

Alternatively, nucleic acid regions of interest can be selected and/or identified using hybridization techniques. Methods for conducting polynucleotide hybridization assays for detection of have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davis, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623.

The present invention also contemplates signal detection of hybridization between ligands in certain preferred aspects. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Patent application 60/364,731 and in PCT Application PCT/US99/06097 (published as WO99/47964).

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application 60/364,731 and in PCT Application PCT/US99/06097 (published as WO99/47964).

### Use of Indices in the Assay Systems of the Invention

In certain aspects, all or a portion of the nucleic acids of interest are directly detected using the described techniques, e.g., sequence determination or hybridization. In certain aspects, however, the nucleic acids of interest are associated with one or more indices that are identifying for a selected nucleic acid region or a particular sample being analyzed. The detection of the one or more indices can serve as a surrogate detection mechanism of the selected nucleic acid region, or as confirmation of the presence of a particular selected nucleic acid region if both the sequence of the index and the sequence of the nucleic acid region itself are determined. These indices are preferably associated with the selected nucleic acids during an amplification step using primers that comprise both the index and sequence regions that specifically hybridize to the nucleic acid region.

In one example, the primers used for selective amplification of a nucleic acid region are designed to provide a locus index between the region complementary to a locus of interest and a universal amplification priming site. The locus index is unique for each selected nucleic acid region and representative of a locus on a chromosome of interest or reference chromosome, so that quantification of the locus index in a sample provides quantification data for the locus and the particular chromosome containing the locus.

In another example, the primers used for amplification of a selected nucleic acid region are designed to provide an allele index between the region complementary to a locus of interest and a universal amplification priming site. The allele index is unique for particular alleles of a selected nucleic acid region and representative of a locus variation present on a chromosome of interest or reference chromosome, so that quantification of the allele index in a sample provides quantification data for the allele and the summation of the allelic indices for a particular locus provides quantification data for both the locus and the particular chromosome containing the locus.

In another aspect, the primers used for amplification of the selected nucleic acid regions to be analyzed for a mixed sample are designed to provide an identification index between the region complementary to a locus of interest and a universal amplification priming site. In such an aspect, a sufficient number of identification indices are present to uniquely identify each selected nucleic acid region in the sample. Each nucleic acid region to be analyzed is associated with a unique identification index, so that the identification index is uniquely associated with the selected nucleic acid region. Quantification of the identification index in a sample provides quantification data for the associated selected nucleic acid region and the chromosome corresponding to the selected nucleic acid region. The identification locus may also be used to detect any amplification bias that occurs downstream of the initial isolation of the selected nucleic acid regions from a sample.

In certain aspects, only the locus index and/or the identification index (if present) are detected and used to quantify the selected nucleic acid regions in a sample. In another aspect, a count of the number of times each locus index occurs with a unique identification index is done to determine the relative frequency of a selected nucleic acid region in a sample.

In some aspects, indices representative of the sample from which a nucleic acid is isolated are used to identify the source of the nucleic acid in a multiplexed assay system. In such aspects, the nucleic acids are uniquely identified with the sample index. Those uniquely identified oligonucleotides may then be combined into a single reaction vessel with nucleic acids from other samples prior to sequencing. The sequencing data is first segregated by each unique sample index prior to determining the frequency of each target locus for each sample and prior to determining whether there is a chromosomal abnormality for each sample. For detection, the sample indices, the locus indices, and the identification indices (if present) are sequenced.

In aspects of the invention using indices, the selective amplification primers are preferably designed so that indices comprising identifying information are coded at one or both ends of the primer. Alternatively, the indices and universal amplification sequences can be added to the selectively amplified nucleic acids following initial selective amplification.

The indices are non-complementary but unique sequences used within the primer to provide information relevant to the selective nucleic acid region that is isolated and/or amplified using the primer. The advantage of this is that information on the presence and quantity of the selected nucleic acid region can be obtained without the need to determine the actual sequence itself, although in certain aspects it may be desirable to do so. Generally, however, the ability to identify and quantify a selected nucleic acid region through identification of one or more indices will decrease the length of sequencing required as the loci information is captured at the 3′ or 5′ end of the isolated selected nucleic acid region. Use of indices identification as a surrogate for identification of selected nucleic acid regions may also reduce error since longer sequencing reads are more prone to the introduction or error.

In addition to locus indices, allele indices and identification indices, additional indices can be introduced to primers to assist in the multiplexing of samples. For example, correction indices which identify experimental error (e.g., errors introduced during amplification or sequence determination) can be used to identify potential discrepancies in experimental procedures and/or detection methods in the assay systems. The order and placement of these indices, as well as the length of these indices, can vary, and they can be used in various combinations.

The primers used for identification and quantification of a selected nucleic acid region may be associated with regions complementary to the 5′ of the selected nucleic acid region, or in certain amplification regimes the indices may be present on one or both of a set of amplification primers which comprise sequences complementary to the sequences of the selected nucleic acid region. The primers can be used to multiplex the analysis of multiple selected nucleic acid regions to be analyzed within a sample, and can be used either in solution or on a solid substrate, e.g., on a microarray or on a bead. These primers may be used for linear replication or amplification, or they may create circular constructs for further analysis.

### Variation Minimization Within and Between Samples

One challenge with the detection of chromosomal abnormalities in a mixed sample is that often the DNA from the cell type with the putative chromosomal abnormality is present in much lower abundance than the DNA from normal cell type. In the case of a mixed maternal sample containing fetal and maternal cell free DNA, the cell free fetal DNA as a percentage of the total cell free DNA may vary from less than one to forty percent, and most commonly is present at or below twenty percent and frequently at or below ten percent. In the detection of an aneuploidy such as Trisomy 21 (Down Syndrome) in the fetal DNA of such mixed maternal sample, the relative increase in Chromosome 21 is 50% in the fetal DNA and thus as a percentage of the total DNA in a mixed sample where, as an example, the fetal DNA is 5% of the total, the increase in Chromosome 21 as a percentage of the total is 2.5%. If one is to detect this difference robustly through the methods described herein, the variation in the measurement of Chromosome 21 has to be much less than the percent increase of Chromosome 21.

The variation between levels found between samples and/or for nucleic acid regions within a sample may be minimized in a combination of analytical methods, many of which are described in this application. For instance, variation is lessened by using an internal reference in the assay. An example of an internal reference is the use of a chromosome present in a “normal” abundance (e.g., disomy for an autosome) to compare against a chromosome present in putatively abnormal abundance, such as aneuploidy, in the same sample. While the use of one such “normal” chromosome as a reference chromosome may be sufficient, it is also possible to use many normal chromosomes as the internal reference chromosomes to increase the statistical power of the quantification.

One method of using an internal reference is to calculate a ratio of abundance of the putatively abnormal chromosomes to the abundance of the normal chromosomes in a sample, called a chromosomal ratio. In calculating the chromosomal ratio, the abundance or counts of each of the nucleic acid regions for each chromosome are summed together to calculate the total counts for each chromosome. The total counts for one chromosome are then divided by the total counts for a different chromosome to create a chromosomal ratio for those two chromosomes.

Alternatively, a chromosomal ratio for each chromosome may be calculated by first summing the counts of each of the nucleic acid regions for each chromosome, and then dividing the sum for one chromosome by the total sum for two or more chromosomes. Once calculated, the chromosomal ratio is then compared to the average chromosomal ratio from a normal population.

The average may be the mean, median, mode or other average, with or without normalization and exclusion of outlier data. In a preferred aspect, the mean is used. In developing the data set for the chromosomal ratio from the normal population, the normal variation of the measured chromosomes is calculated. This variation may be expressed a number of ways, most typically as the coefficient of variation, or CV. When the chromosomal ratio from the sample is compared to the average chromosomal ratio from a normal population, if the chromosomal ratio for the sample falls statistically outside of the average chromosomal ratio for the normal population, the sample contains an aneuploidy. The criteria for setting the statistical threshold to declare an aneuploidy depend upon the variation in the measurement of the chromosomal ratio and the acceptable false positive and false negative rates for the desired assay. In general, this threshold may be a multiple of the variation observed in the chromosomal ratio. In one example, this threshold is three or more times the variation of the chromosomal ratio. In another example, it is four or more times the variation of the chromosomal ratio. In another example it is five or more times the variation of the chromosomal ratio. In another example it is six or more times the variation of the chromosomal ratio. In the example above, the chromosomal ratio is determined by summing the counts of nucleic acid regions by chromosome. Typically, the same number of nucleic acid regions for each chromosome is used. An alternative method for generating the chromosomal ratio would be to calculate the average counts for the nucleic acid regions for each chromosome. The average may be any estimate of the mean, median or mode, although typically an average is used. The average may be the mean of all counts or some variation such as a trimmed or weighted average. Once the average counts for each chromosome have been calculated, the average counts for each chromosome may be divided by the other to obtain a chromosomal ratio between two chromosomes, the average counts for each chromosome may be divided by the sum of the averages for all measured chromosomes to obtain a chromosomal ratio for each chromosome as described above. As highlighted above, the ability to detect an aneuploidy in a mixed sample where the putative DNA is in low relative abundance depends greatly on the variation in the measurements of different nucleic acid regions in the assay. Numerous analytical methods can be used which reduce this variation and thus improve the sensitivity of this method to detect aneuploidy.

One method for reducing variability of the assay is to increase the number of nucleic acid regions used to calculate the abundance of the chromosomes. In general, if the measured variation of a single nucleic acid region of a chromosome is X % and Y different nucleic acid regions are measured on the same chromosome, the variation of the measurement of the chromosomal abundance calculated by summing or averaging the abundance of each nucleic acid region on that chromosome will be approximately X % divided by Y^{1/2}. Stated differently, the variation of the measurement of the chromosome abundance would be approximately the average variation of the measurement of each nucleic acid region's abundance divided by the square root of the number of nucleic acid regions.

In a preferred aspect of this invention, the number of nucleic acid regions measured for each chromosome is at least 24. In another preferred aspect of this invention, the number of nucleic acid regions measured for each chromosome is at least 48. In another preferred aspect of this invention, the number of nucleic acid regions measured for each chromosome is at least 100. In another preferred aspect of this invention the number of nucleic acid regions measured for each chromosome is at least 200. There is incremental cost to measuring each nucleic acid region and thus it is important to minimize the number of each nucleic acid region. In a preferred aspect of this invention, the number of nucleic acid regions measured for each chromosome is less than 2000. In a preferred aspect of this invention, the number of nucleic acid regions measured for each chromosome is less than 1000. In a most preferred aspect of this invention, the number of nucleic acid regions measured for each chromosome is at least 48 and less than 1000. In one aspect, following the measurement of abundance for each nucleic acid region, a subset of the nucleic acid regions may be used to determine the presence or absence of aneuploidy. There are many standard methods for choosing the subset of nucleic acid regions. These methods include outlier exclusion, where the nucleic acid regions with detected levels below and/or above a certain percentile are discarded from the analysis. In one aspect, the percentile may be the lowest and highest 5% as measured by abundance. In another aspect, the percentile may be the lowest and highest 10% as measured by abundance. In another aspect, the percentile may be the lowest and highest 25% as measured by abundance.

Another method for choosing the subset of nucleic acid regions include the elimination of regions that fall outside of some statistical limit. For instance, regions that fall outside of one or more standard deviations of the mean abundance may be removed from the analysis. Another method for choosing the subset of nucleic acid regions may be to compare the relative abundance of a nucleic acid region to the expected abundance of the same nucleic acid region in a healthy population and discard any nucleic acid regions that fail the expectation test. To further minimize the variation in the assay, the number of times each nucleic acid region is measured may be increased. As discussed, in contrast to the random methods of detecting aneuploidy where the genome is measured on average less than once, the assay systems of the present invention intentionally measures each nucleic acid region multiple times. In general, when counting events, the variation in the counting is determined by Poisson statistics, and the counting variation is typically equal to one divided by the square root of the number of counts. In a preferred aspect of the invention, the nucleic acid regions are each measured on average at least 100 times. In a preferred aspect to the invention, the nucleic acid regions are each measured on average at least 500 times. In a preferred aspect to the invention, the nucleic acid regions are each measured on average at least 1000 times. In a preferred aspect to the invention, the nucleic acid regions are each measured on average at least 2000 times. In a preferred aspect to the invention, the nucleic acid regions are each measured on average at least 5000 times.

In another aspect, subsets of loci can be chosen randomly using sufficient numbers to yield a statistically significant result in determining whether a chromosomal abnormality exists. Multiple analyses of different subsets of loci can be performed within a mixed sample to yield more statistical power. In this example, it may or may not be necessary to remove or eliminate any loci prior to the random analysis. For example, if there are 100 selected regions for chromosome 21 and 100 selected regions for chromosome 18, a series of analyses could be performed that evaluate fewer than 100 regions for each of the chromosomes.

In addition to the methods above for reducing variation in the assay, other analytical techniques, many of which are described earlier in this application, may be used in combination. In general, the variation in the assay may be reduced when all of the nucleic acid regions for each sample are interrogated in a single reaction in a single vessel. Similarly, the variation in the assay may be reduced when a universal amplification system is used. Furthermore, the variation of the assay may be reduced when the number of cycles of amplification is limited.

### Determination of Fetal DNA Content in Maternal Sample

In certain specific aspects, determining the relative percentage of fetal DNA in a maternal sample may be beneficial in performing the assay system, as it will provide important information on the expected statistical presence of genomic regions and variation from that expectation may be indicative copy number variation associated with insertion, deletions or aneuploidy. This may be especially helpful in circumstances where the level of fetal DNA in a maternal sample is low, as the percent fetal contribution can be used in determining the quantitative statistical significance in the variations of levels of identified nucleic acid regions in a maternal sample. In other aspects, the determining of the relative percent fetal cell free DNA in a maternal sample may be beneficial in estimating the level of certainty or power in detecting a fetal aneuploidy.

In some specific aspects, the relative fetal contribution of maternal DNA at the allele of interest can be compared to the non-maternal contribution at that allele to determine approximate fetal DNA concentration in the sample. In other specific aspects, the relative quantity of solely paternally-derived sequences (e.g., Y-chromosome sequences or paternally-specific polymorphisms) can be used to determine the relative concentration of fetal DNA in a maternal sample.

Another exemplary approach to determining the percent fetal contribution in a maternal sample through the analysis of DNA fragments with different patterns of DNA methylation between fetal and maternal DNA.

Determination of Fetal DNA Content in a Maternal Sample Using Y-Specific Sequences

In circumstances where the fetus is male, percent fetal DNA in a sample can be determined through detection of Y-specific nucleic acids and comparison to calculated maternal DNA content. Quantities of an amplified Y-specific nucleic acid, such as a region from the sex-determining region Y gene (SRY), which is located on the Y chromosome and is thus representative of fetal DNA, can be determined from the sample and compared to one or more amplified genes which are present in both maternal DNA and fetal DNA and which are preferably not from a chromosome believed to potentially be aneuploid in the fetus, e.g., an autosomal region that is not on chromosome 21 or 18. Preferably, this amplification step is performed in parallel with the selective amplification step, although it may be performed either before or after the selective amplification depending on the nature of the multiplexed assay.

In a preferred aspect, the amplified DNA is obtained from cell free DNA by polymerase chain reaction (PCR). Other mechanisms for amplification can be used as well, including those described in more detail herein, as will be apparent to one skilled in the art upon reading the present disclosure.

In particular aspects, the percentage of cell free fetal DNA in the maternal sample can determined by PCR using serially diluted DNA isolated from the maternal sample, which can accurately quantify the number of genomes comprising the amplified genes. For example, if the blood sample contains 100% male fetal DNA, and 1:2 serial dilutions are performed, then on average the SRY signal will disappear 1 dilution before the autosomal signal, since there is 1 copy of the SRY gene and 2 copies of the autosomal gene.

In a specific aspect, the percentage of free fetal DNA in maternal plasma is calculated using the following formula: percentage of free fetal DNA=(No. of copies of SRY gene×2×100)/(No. of copies of autosomal gene), where the number of copies of each gene is determined by observing the highest serial dilution in which the gene was detected. The formula contains a multiplication factor of 2, which is used to normalize for the fact that there is only 1 copy of the SRY gene compared to two copies of the autosomal gene in each genome, fetal or maternal.

Determination of Fetal DNA Content in a Maternal Sample Using Fetal Autosomal Polymorphisms and Genetic Variations

In each maternally-derived sample, the DNA from a fetus will have approximately 50% of its loci inherited from the mother and 50% of the loci inherited from the father. Determining the loci contributed to the fetus from non-maternal sources can allow the estimation of fetal DNA in a maternal sample, and thus provide information used to calculate the statistically significant differences in chromosomal frequencies for chromosomes of interest.

In certain aspects, the determination of fetal polymorphisms requires targeted SNP and/or mutation analysis to identify the presence of fetal DNA in a maternal sample. In some aspects, the use of prior genotyping of the father and mother can be performed. For example, the parents may have undergone such genotype determination for identification of disease markers, e.g., determination of the genotype for disorders such as cystic fibrosis, muscular dystrophy, spinal muscular atrophy or even the status of the RhD gene may be determined. Such difference in polymorphisms, copy number variants or mutations can be used to determine the percentage fetal contribution in a maternal sample.

In one preferred aspect, the percent fetal cell free DNA in a maternal sample can be quantified using multiplexed SNP detection without using prior knowledge of the maternal or paternal genotype. In this aspect, two or more selected polymorphic nucleic acid regions with a known SNP in each region are used. In a preferred aspect, the selected polymorphic nucleic acid regions are located on an autosomal chromosome that is unlikely to be aneuploidy, e.g., Chromosome 6. The selected polymorphic nucleic acid regions from the maternal are amplified. In a preferred aspect, the amplification is universal.

In a preferred embodiment, the selected polymorphic nucleic acid regions are amplified in one reaction in one vessel. Each allele of the selected polymorphic nucleic acid regions in the maternal sample is determined and quantified. In a preferred aspect, high throughput sequencing is used for such determination and quantification. Following sequence determination, loci are identified where the maternal and fetal genotypes are different, e.g., the maternal genotype is homozygous and the fetal genotype is heterozygous. This identification is done by observing a high relative frequency of one allele (>60%) and a low relative frequency (<20% and >0.15%) of the other allele for a particular selected nucleic acid region. The use of multiple loci is particularly advantageous as it reduces the amount of variation in the measurement of the abundance of the alleles. All or a subset of the loci that meet this requirement are used to determine fetal concentration through statistical analysis.

In one aspect, fetal concentration is determined by summing the low frequency alleles from two or more loci together, dividing by the sum of the high and low frequency alleles and multiplying by two. In another aspect, the percent fetal cell free DNA is determined by averaging the low frequency alleles from two or more loci, dividing by the average of the high and low frequency alleles and multiplying by two.

For many alleles, maternal and fetal sequences may be homozygous and identical, and as this information is not distinguishing between maternal and fetal DNA it is not useful in the determination of percent fetal DNA in a maternal sample. The present invention utilizes allelic information where there is a distinguishable difference between the fetal and maternal DNA (e.g., a fetal allele containing at least one allele that differs from the maternal allele) in calculations of percent fetal. Data pertaining to allelic regions that are the same for the maternal and fetal DNA are thus not selected for analysis, or are removed from the pertinent data prior to determination of percentage fetal DNA so as not to swamp out the useful data.

Exemplary methods for quantifying fetal DNA in maternal plasma can be found, e.g., in Chu et al., Prenat Diagn 2010; 30:1226-1229, which is incorporated herein by reference.

In one aspect, selected nucleic acid regions may be excluded if the amount or frequency of the region appears to be an outlier due to experimental error, or from idiopathic genetic bias within a particular sample. In another aspect, selected nucleic acids may undergo statistical or mathematical adjustment such as normalization, standardization, clustering, or transformation prior to summation or averaging. In another aspect, selected nucleic acids may undergo both normalization and data experimental error exclusion prior to summation or averaging.

In a preferred aspect, 12 or more loci are used for the analysis. In another preferred aspect, 24 or more loci are used for the analysis. In another preferred aspect, 48 or more loci are used for the analysis. In another aspect, one or more indices are used to identify the sample, the locus, the allele or the identification of the nucleic acid.

In one preferred aspect, the percentage fetal contribution in a maternal sample can be quantified using tandem SNP detection in the maternal and fetal alleles. Techniques for identifying tandem SNPs in DNA extracted from a maternal sample are disclosed in Mitchell et al, U.S. Pat. No. 7,799,531 and U.S. patent application Ser. Nos. 12/581,070, 12/581,083, 12/689,924, and 12/850,588. These describe the differentiation of fetal and maternal loci through detection of at least one tandem single nucleotide polymorphism (SNP) in a maternal sample that has a different haplotype between the fetal and maternal genome. Identification and quantification of these haplotypes can be performed directly on the maternal sample, as described in the Mitchell et al. disclosures, and used to determine the percent fetal contribution in the maternal sample.

Determination of Fetal DNA Content in a Maternal Sample Using Epigenetic Allelic Ratios

Certain genes have been identified as having epigenetic differences between the placenta and maternal blood cells, and such genes are candidate loci for fetal DNA markers in a maternal sample. See, e.g., Chim S S C, et al. Proc Natl Acad Sci USA (2005); 102:14753-14758. These loci, which are unmethylated in the placenta but not in maternal blood cells, can be readily detected in maternal plasma and were confirmed to be fetus specific. Unmethylated fetal DNA can be amplified with high specificity by use of methylation-specific PCR (MSP) even when such fetal DNA molecules were present among an excess of background plasma DNA of maternal origin. The comparison of methylated and unmethylated amplification products in a maternal sample can be used to quantify the percent fetal DNA contribution to the maternal sample by calculating the epigenetic allelic ratio for one or more of such sequences known to be differentially regulated by methylation in the fetal DNA as compared to maternal DNA.

To determine methylation status of nucleic acids in a maternal sample, the nucleic acids of the sample are subjected to bisulfite conversion of the samples and then subjected them to MSP, followed by allele-specific primer extension. Conventional methods for such bisulphite conversion include, but are not limited to, use of commercially available kits such as the Methylamp™ DNA Modification Kit (Epigentek, Brooklyn, N.Y.). Allelic frequencies and ratios can be directly calculated and exported from the data to determine the relative percentage of fetal DNA in the maternal sample.

### Use of Percent Fetal Cell Free DNA to Optimize Aneuploidy Detection

Once the percent fetal cell free DNA has been calculated, this data may be combined with methods for aneuploidy detection to determine the likelihood that a maternal sample may contain an aneuploidy. In one aspect, an aneuploidy detection methods that utilizes analysis of random DNA segments is used, such as that described in, e.g., Quake, U.S. patent application Ser. No. 11/701,686; Shoemaker et al., U.S. patent application Ser. No. 12/230,628. In a preferred aspect, aneuploidy detection methods that utilize analysis of selected nucleic acid regions are used. In this aspect, the percent fetal cell free DNA for a sample is calculated. The chromosomal ratio for that sample, a chromosomal ratio for the normal population and a variation for the chromosomal ratio for the normal population is determined, as described herein.

In one preferred aspect, the chromosomal ratio and its variation for the normal population are determined from normal samples that have a similar percentage of fetal DNA. An expected aneuploidy chromosomal ratio for a DNA sample with that percent fetal cell free DNA is calculated by adding the percent contribution from the aneuploidy chromosome. The chromosomal ratio for the sample may then be compared to the chromosomal ratio for the normal population and to the expected aneuploidy chromosomal ratio to determine statistically, using the variation of the chromosomal ratio, to determine if the sample is more likely normal or aneuploidy, and the statistical probability that it is one or the other.

In a preferred aspect, the selected regions of a mixed sample include both regions for determination of fetal DNA content as well as non-polymorphic regions from two or more chromosomes to detect a fetal chromosomal abnormality in a single reaction. The single reaction helps to minimize the risk of contamination or bias that may be introduced during various steps in the assay system which may otherwise skew results when utilizing fetal DNA content to help determine the presence or absence of a chromosomal abnormality.

In other aspects, a selected region or regions may be utilized both for determination of fetal DNA content as well as detection of fetal chromosomal abnormalities. The alleles for selected regions can be used to determine fetal DNA content and these same selected regions can then be used to detect fetal chromosomal abnormalities ignoring the allelic information. Utilizing the same regions for both fetal DNA content and detection of chromosomal abnormalities may further help minimize any bias due to experimental error or contamination.

### Detection of Genetic Mutations

In certain aspects, the assay system of the invention detects both fetal aneuploidies and other genetic alterations (including chromosomal abnormalities) in specific loci of interest. Such additional genetic alterations include, but are not limited to, deletion mutations, insertion mutations, copy number polymorphisms, copy number variants, chromosome 22q11 deletion syndrome, 11q deletion syndrome on chromosome 11, 8p deletion syndrome on chromosome 8, and the like. Generally, at least two target nucleic acid sequences present on the same or separate chromosomes are analyzed, and at least one of the target sequences is associated with the fetal allelic abnormality. The sequences of the two target sequences and number of copies of the two target sequences are then compared to determine whether the chromosomal abnormality is present, and if so, the nature of the abnormality.

While much of the description contained herein describes detecting aneuploidy by counting the abundance of nucleic acid regions on one or more putative aneuploid chromosomes and the abundance of nucleic acid regions on one or more normal chromosomes, the same techniques may be used to detect copy number variations where such copy number variation occurs on only a portion of a chromosome. In this detection of the copy number variations, multiple nucleic acid regions within the putative copy number variation location are compared to multiple nucleic acid regions outside of the putative copy number variation location. Other aspects of the invention described for aneuploidy may then be used for the detection of copy number variation. For instance, one may detect a chromosome 22q11 deletion syndrome in a fetus in a mixed maternal sample by selecting two or more nucleic regions within the 22q11 deletion and two or more nucleic acid regions outside of the 22q11 deletion. The nucleic acid regions outside of the 22q11 deletion may be on another region of Chromosome 22 or may be on a completely different chromosome. The abundance of each nucleic acid regions is determined by the methods described in this application.

The nucleic acid regions within the deletion are then summed as are the nucleic acid regions outside of the deletion. These sums are then compared to each other to determine the presence or absence of a deletion. Optionally, the sums are put into a ratio and that ratio may be compared to an average ratio created from a normal population. When the ratio for a sample falls statistically outside of an expected ratio, the deletion is detected. The threshold for the detection of a deletion may be four or more times the variation calculated in the normal population.

### Use of Other Fetal Detection Methods

In certain aspects of the invention, the methods of the invention can be used in conjunction with detection of other known risk factors (e.g., maternal age, family history, maternal or paternal genetic information) and/or means for detecting fetal abnormalities, and preferably with other relatively non-invasive diagnostic mechanisms of fetal abnormalities (e.g., measurements of one or more biochemical markers in a maternal sample and/or measurements or structural detection from an ultrasound scan). The combined use of these risk factors and diagnostic mechanisms with the methods of the invention can provide an improved risk determination of fetal abnormality, and in particular the presence or absence of a known genetic mutation such as a trisomy.

Thus, in some preferred aspects the results obtained in the assay systems of the invention are combined with the results from biochemical detection of risk factors, ultrasound detection of risk factors, or other risk determinants of fetal abnormalities.

In some specific aspects, the results obtained in the assay systems of the invention are combined with detection of biochemical markers associated with an increased risk of fetal abnormality. The biochemical markers can be determined based on a sample comprising maternal blood, serum, plasma or urine. Such biochemical markers include but are not limited to free Beta hCG, pregnancy-associated plasma protein A (PAPP-A), maternal blood alpha-fetoprotein, maternal blood hCG, maternal blood unconjugated estriol, maternal blood dimeric inhibin A, maternal urine total estriol, maternal urine beta core fragment, maternal urine hyperglycosylated hCG, maternal blood hyperglycosylated hCG, and inhibin A (preferably dimeric inhibin A). In some aspects, the additional assessment mechanism is multimarker analysis, such as that described in Orlandi et al., U.S. Pat. No. 7,315,787 or Wald et al. U.S. Pat. No. 6,573,103. Detection of presence and/or levels of these and other markers can be combined with the results from assay systems of the invention to provide a final result to the patient.

In other specific aspects, the results obtained in the assay systems of the invention are combined with the results obtained from ultrasound images, including but are not limited to: nuchal translucency (NT) thickness or edema, nuchal fold thickness, abnormality of the venous system (including the ductus venosus, the portal and hepatic veins and inferior vena cava), absent or hypoplastic nasal bone, femur length, humerus length, hyperechogenic bowel, renal pyelectasis, echogenic foci in the heart, fetal heart rate, and certain cardiac abnormalities. In specific aspects, the additional assessment of fetal abnormality is performed though shape analysis, such as described in U.S. Pat. Nos. 7,780,600 and 7,244,233. In a specific aspect, the additional assessment is based on the determination of landmarks based on images, as described in U.S. Pat. No. 7,343,190. Detection of these and other physical parameters can be combined with the results from assay systems of the invention to provide a final result to the patient.

Most screening markers and physical characteristics are known to vary with gestational age. To take account of this variation each marker level may be expressed as a multiple of the median level (MoM) for unaffected pregnancies of the same gestational age. Especially, for markers derived from ultrasound scans, crown-rump length (CRL) or biparietal diameter (BPD) measurement are alternative measures of gestational age. MoMs may be adjusted in a known way to take account of factors which are known to affect marker levels, such as maternal weight, ethnic group, diabetic status and the number of fetuses carried.

Use of the above techniques can be performed at a single stage of pregnancy or obtained sequentially at two or more different stages of pregnancy. These marker levels can also be interpreted in combination with variables maternal such as maternal age, weight, ethnicity, etc. to derive a risk estimate. The estimation of risk is conducted using standard statistical techniques. For example, known methods are described in Wald N J et al., BMJ (1992); 305(6850):391-4; Wald N J et al (1988) BMJ 297:883-887 and in Royston P, Thompson S G Stat Med. (1992) 11(2):257-68.

### Detection of Other Agents or Risk Factors in Mixed Sample

Given the multiplexed nature of the assay systems of the invention, in certain aspects it may be beneficial to utilize the assay to detect other nucleic acids that could pose a risk to the health of the subject(s) or otherwise impact on clinical decisions about the treatment or prognostic outcome for a subject. Such nucleic acids could include but are not limited to indicators of disease or risk such as maternal alleles, polymorphisms, or somatic mutations known to present a risk for maternal or fetal health. Such indicators include, but are not limited to, genes associated with Rh status; mutations or polymorphisms associated with diseases such as diabetes, hyperlipidemia, hypercholesterolemia, blood disorders such as sickle cell anemia, hemophilia or thalassemia, cardiac conditions, etc.; exogenous nucleic acids associated with active or latent infections; somatic mutations or copy number variations associated with autoimmune disorders or malignancies (e.g., breast cancer), or any other health issue that may impact on the subject, and in particular on the clinical options that may be available in the treatment and/or prevention of health risks in a subject based on the outcome of the assay results.

Accordingly, as the preferred assay systems of the invention are highly multiplexed and able to interrogate hundreds or even thousands of nucleic acids within a mixed sample, in certain aspects it is desirable to interrogate the sample for nucleic acid markers within the mixed sample, e.g., nucleic acids associated with genetic risk or that identify the presence or absence of infectious organisms. Thus, in certain aspects, the assay systems provide detection of such nucleic acids in conjunction with the detection of nucleic acids for copy number determination within a mixed sample.

For example, in certain mixed samples of interest, including maternal samples, samples from subjects with autoimmune disease, and samples from patients undergoing chemotherapy, the immune suppression of the subject may increase the risk for the disease due to changes in the subject's immune system. Detection of exogenous agents in a mixed sample may be indicative of exposure to and infection by an infectious agent, and this finding have an impact on patient care or management of an infectious disease for which a subject tests positively for such infectious agent.

Specifically, changes in immunity and physiology during pregnancy may make pregnant women more susceptible to or more severely affected by infectious diseases. In fact, pregnancy itself may be a risk factor for acquiring certain infectious diseases, such as toxoplasmosis, Hansen disease, and listeriosis. In addition, for pregnant women or subjects with suppressed immune systems, certain infectious diseases such as influenza and varicella may have a more severe clinical course, increased complication rate, and higher case-fatality rate. Identification of infectious disease agents may therefore allow better treatment for maternal disease during pregnancy, leading to a better overall outcome for both mother and fetus.

In addition, certain infectious agents can be passed to the fetus via vertical transmission, i.e. spread of infections from mother to baby. These infections may occur while the fetus is still in the uterus, during labor and delivery, or after delivery (such as while breastfeeding).

Thus, is some preferred aspects, the assay system may include detection of exogenous sequences, e.g., sequences from infectious organisms that may have an adverse effect on the health and/or viability of the fetus or infant, in order to protect maternal, fetal, and or infant health.

Exemplary infections which can be spread via vertical transmission, and which can be tested for using the assay methods of the invention, include but are not limited to congenital infections, perinatal infections and postnatal infections.

Congenital infections are passed in utero by crossing the placenta to infect the fetus. Many infectious microbes can cause congenital infections, leading to problems in fetal development or even death. TORCH is an acronym for several of the more common congenital infections. These are: toxoplasmosis, other infections (e.g., syphilis, hepatitis B, Coxsackie virus, Epstein-Ban virus, varicella-zoster virus (chicken pox), and human parvovirus B19 (fifth disease)), rubella, cytomegalovirus (CMV), and herpes simplex virus.

Perinatal infections refer to infections that occur as the baby moves through an infected birth canal or through contamination with fecal matter during delivery. These infections can include, but are not limited to, sexually-transmitted diseases (e.g., gonorrhea, chlamydia, herpes simplex virus, human papilloma virus, etc.) CMV, and Group B Streptococci (GBS).

Infections spread from mother to baby following delivery are known as postnatal infections. These infections can be spread during breastfeeding through infectious microbes found in the mother's breast milk. Some examples of postnatal infections are CMV, Human immunodeficiency virus (HIV), Hepatitis C Virus (HCV), and GBS.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric.

### Example 1

### Sample Procurement

Subjects were prospectively enrolled upon providing informed consent, under protocols approved by institutional review boards. Subjects were required to be at least 18 years of age, at least 10 weeks gestational age, and to have singleton pregnancies. A subset of enrolled subjects, consisting of 250 women with disomic pregnancies, 72 with T21 pregnancies, and 16 with T18 pregnancies, was selected for inclusion in this study. The subjects were randomized into a first cohort consisting of 127 disomic pregnancies, 36 T21 pregnancies, and 8 T18 pregnancies, and a second cohort consisting of 123 disomic pregnancies, 36 T21 pregnancies, and 8 T18 pregnancies. The trisomy status of each pregnancy was confirmed by invasive testing (fluorescent in-situ hybridization and/or karyotype analysis). The trisomy status of the first cohort was known at the time of analysis; in the second cohort, the trisomy status was kept blinded until after analysis.

8 mL blood per subject was collected into a Cell-free DNA tube (Streck, Omaha, Nebr.) and stored at room temperature for up to 3 days. Plasma was isolated from blood via double centrifugation and stored at −20° C. for up to a year. cfDNA was isolated from plasma using Viral NA DNA purification beads (Life Technologies, Carlsbad, Calif.), biotinylated, immobilized on MyOne C1 streptavidin beads (Life Technologies, Carlsbad, Calif.).

### Example 2

### Design of Primer Pairs for Amplification of Selected Genomic Regions

Assays were designed based on human genomic sequences, and each interrogation consisted of two fixed sequence oligos per selected nucleic acid region interrogated in the assay. The first oligo, complementary to the 3′ region of a genomic region, comprised the following sequential (5′ to 3′) oligo elements: a universal PCR priming sequence common to all assays: TACACCGGCGTTATGCGTCGAGAC (SEQ ID NO:1); a nine nucleotide identification code specific to the selected genomic region; a hybridization breaking nucleotide which is different from the corresponding base in the genomic region; and a 20-24 bp sequence complementary to the selected genomic region. These first oligos were designed for each selected nucleic acid to provide a predicted uniform Tₘ with a two degree variation across all interrogations in the assay set.

The second fixed sequence oligo, complementary to the 5′ region of the genomic loci, comprised the following sequential (5′ to 3′) elements: a 20-24b sequence complimentary to the 5′ region in the genomic locus; a hybridization breaking nucleotide which was different from the corresponding base in the genomic locus; and a universal PCR priming sequence which was common to all third oligos in the assay set: ATTGCGGGGACCGATGATCGCGTC (SEQ ID NO:2). This second oligo was designed for each selected nucleic acid to provide a predicted uniform Tₘ with a two degree variation across all interrogations in the assay set that was substantially the same Tₘ range as the first oligo set.

All oligonucleotides were synthesized using conventional solid-phase chemistry. The first and bridging oligonucleotides were synthesized with 5′phosphate moieties to enable ligation to 3′ hydroxyl termini of adjacent oligonucleotides. An equimolar pool of sets of the first and third oligonucleotides used for all interrogations in the multiplexed assay was created, and a separate equimolar pool of all bridging oligonucleotides was created to allow for separate hybridization reactions.

### Example 3

### Design of Padlock Probes for Amplification of Selected Genomic Regions

Assays are designed based on human genomic sequences, and each interrogation consists of a single oligo with two regions complementary to selected nucleic acid region interrogated in the assay. The 5′ end of the padlock probe, complementary to the 3′ region of a genomic region, comprises the following sequential (5′ to 3′) oligo elements: a universal PCR priming sequence common to all assays (TACACCGGCGTTATGCGTCGAGAC (SEQ ID NO:1)); a nine nucleotide identification code specific to the selected loci; a 9 base locus- or locus/allele-specific sequence that acts as a locus code; a hybridization breaking nucleotide which is different from the corresponding base in the genomic locus; and a 20-24 bp sequence complementary to the selected genomic region. The 3′ end of the padlock probe, complementary to the 5′ region of the genomic loci, comprises the following sequential (5′ to 3′) elements: a 20-24 b sequence complimentary to the 5′ region in the genomic locus; a hybridization breaking nucleotide which was different from the corresponding base in the genomic locus; and a universal PCR priming sequence common to all third oligos in the assay set (ATTGCGGGGACCGATGATCGCGTC (SEQ ID NO:2)). The padlock probes are designed for each selected nucleic acid to provide a predicted uniform Tₘ with a two degree variation across all interrogations in the assay set.

### Example 4

### Determination of Chromosome Proportion in a First Patient Cohort

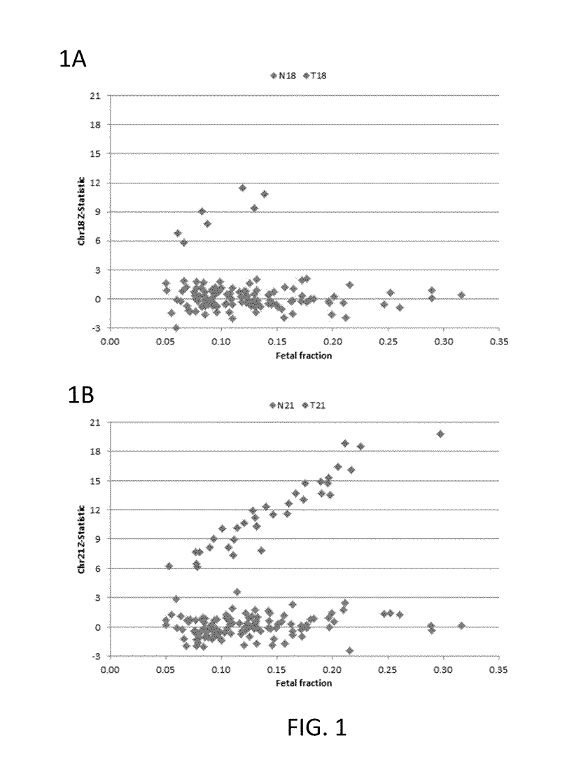

For initial selection of loci to be used for aneuploidy detection, a set of subjects whose aneuploidy status was known was evaluated. This first cohort consisted of 121 normal, 35 T21, and 7 T18 pregnancies. Chromosome proportion Z Statistics were determined for these samples, as illustrated in FIGS. 1A and 1B. 120/121 (99.2%) disomic samples had Z Statistics<3. One disomic sample had a chr21 Z Statistic of 3.5. 35/35 (100%) T21 and 7/7 (100%) T18 samples had chromosome proportion Z Statistics>3. Thus, using Z Statistic analysis, the assay system exhibited 99.2% specificity and 100% sensitivity for T21, and 100% specificity and 100% sensitivity for T18.

The regions were selectively amplified from the cfDNA prepared as described in Example 1 using oligonucleotides complementary designed as described in Example 2 and a third bridging oligo using methods as described in U.S. Appln No. 13/013,732, which is incorporated by reference. A selective amplification product was generated from each subject sample. Following the initial selection of the genomic regions using the designed oligonucleotides, the amplification products were eluted from the cfDNA and further amplified using universal PCR primers complementary to the universal primer sequences of the oligonucleotides. Briefly, a 50 μl universal PCR reaction consisting of 25 μL eluted amplification product plus 1×Pfusion buffer (Finnzymes, Finland), 1M Betaine, 400 nM each dNTP, 1 U Pfusion error-correcting thermostable DNA polymerase, and the universal primer pairs.

PCR products from 96 independent samples was pooled and used as template for cluster amplification on a single lane of a TruSeq v2 SR flow slide (Illumina, San Diego, Calif.). The slide was processed on an Illumina HiSeq™ 2000 to produce a 56 base locus-specific sequence and a 7 base sample tag sequence from an average of 1.18 million (M) clusters/sample. Locus specific reads were compared to expected locus sequences. An average of 1.15M (97%) reads had fewer than 3 mismatches with expected locus sequences, resulting in an average of 854 reads/locus/sample.

Sequence counts were normalized by systematically removing sample and assay biases. Sequence counts follow a log normal distribution, so biases were estimated using median polish on log transformed counts. Tukey, J W. Exploratory Data Analysis. Reading Mass.: Addison-Wesley. 1977; Irizarry R A et al., Nucleic Acids Res 2003; 31(4): e15. A chromosome 21 proportion metric was computed for each sample as the mean of counts for selected chromosome 21 loci divided by the sum of the mean of counts for selected chromosome 21 loci and the mean of counts for all 576 chromosome 18 loci. A chromosome 18 proportion metric was similarly calculated for each sample. A standard Z test of proportions was used to compute Z Statistics,

$${Z}_{j} = \frac{{p}_{j} - {p}_{0}}{\sqrt{\frac{{p}_{0} ( 1 - {p}_{0} )}{{n}_{j}}}}$$

where pj is the observed proportion for a given chromosome of interest in a given sample j, p0 is the expected proportion for the given test chromosome calculated as the median pj, and nj is the denominator of the proportion metric. Z Statistic standardization was performed using iterative censoring on each lane of 96 samples. At each iteration, the samples falling outside of 3 median absolute deviations were removed. After 10 iterations, mean and standard deviation were calculated using only the uncensored samples. All samples were then standardized against this mean and standard deviation. The Kolmogorov-Smirnov test (Conover W J. Practical Nonparametric Statistics. New York: John Wiley & Sons. 1971; p. 295-301) and Shapiro-Wilk's test (Royston P. Applied Statistics 1982; 31:115-124) were used to establish the normality of the uncensored samples' Z Statistics.

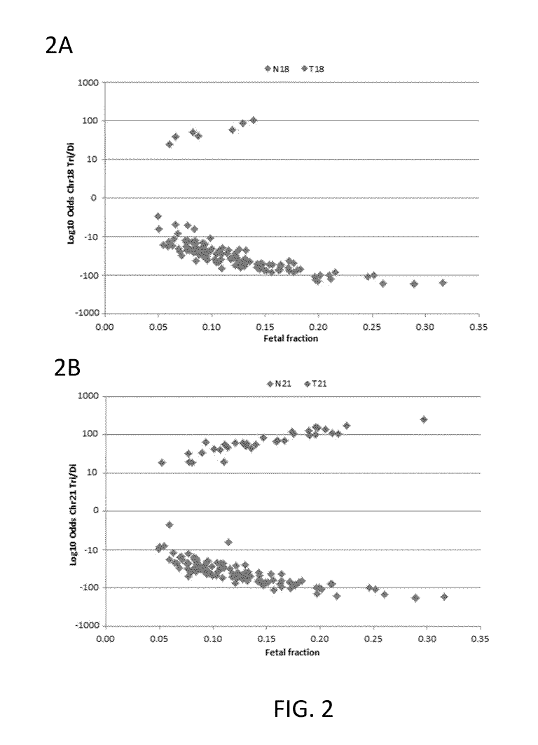

A principal determinant of the chromosome proportion response to aneuploidy is the fraction of fetal DNA in the sample. In order to measure fetal fraction reliably, 192 DANSR assays targeting SNPs were incorporated into a multiplex assay pool. By measuring fetal fraction and chromosome proportion in the same reaction, estimates of fetal fraction from polymorphic assays closely represented fetal fraction in the non-polymorphic assays used to assess chromosome proportion. Fetal fraction exhibited a strong correlation (R2>0.90) with the chromosome proportion Z Statistic in trisomic pregnancies (FIGS. 1A and 1B). Importantly, the Z Statistic was not responsive to fetal fraction in normal pregnancies, reflecting a major limitation of the Z Statistic metric: samples with low Z Statistic values arise from both euploid samples and aneuploid samples with modest fetal fraction. The odds of trisomy versus disomy of chr18 and chr21 were then determined in each sample within the first cohort (FIGS. 2A and 2B).

### Example 5

### Determination of Chromosome Proportion in a Second Patient Cohort

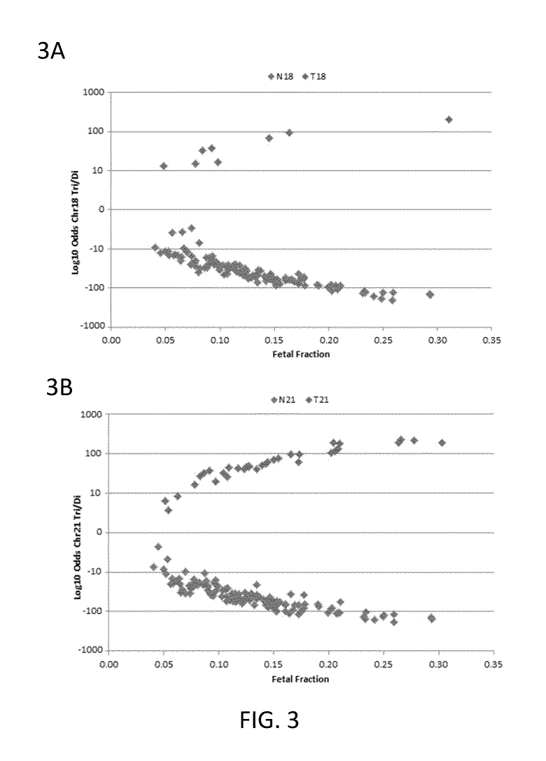

In order to test the performance of the assay systems in an independent set of subjects, a second blinded cohort consisting of 123 normal, 36 T21, and 8 T18 pregnancies was assayed as described in Example 4. All samples passed QC criteria and were assigned odds scores for chr18 and chr21 (FIGS. 3A and 3B). As above, the assay and corresponding analysis correctly discriminated all trisomy from disomy subjects. The difference between the lowest aneuploid odds and the highest euploid odds was 103.9. All 36 T21 and 8 T18 samples had trisomy odds exceeding 102.67 (>99.8% risk of trisomy).

### Example 6

### Determination of Percent Fetal DNA Levels in Maternal Samples

One exemplary assay system of the invention was designed comprising 480 separate interrogations, each utilizing the detection of different loci in a maternal sample. The initial example utilized a determination of percent fetal DNA in subjects carrying a male fetus, and so loci on the Y chromosome were utilized as well as loci containing a paternally-inherited fetal SNP that is different from the maternal sequence.

Specifically, 480 selected nucleic acids were interrogated using the assay system. The 480 selected nucleic acids comprised 48 sequence-specific interrogations of nucleic acids corresponding to loci on chromosome Y, 192 sequence-specific interrogations of nucleic acids corresponding to loci on chromosome 21, 192 sequence-specific interrogations of selected nucleic acids corresponding to loci on chromosome 18, and 144 sequence-specific interrogations of selected nucleic acids corresponding to polymorphic loci on chromosomes 1-16 which. These assays were designed based on human genomic sequences, and each interrogation used three oligos per selected nucleic acid interrogated in the assay.

The first oligo used for each interrogation was complementary to the 3′ region of the selected genomic region, and comprised the following sequential (5′ to 3′) oligo elements: a universal PCR priming sequence common to all assays: TACACCGGCGTTATGCGTCGAGAC (SEQ ID NO:1); an identification code specific to the selected loci comprising nine nucleotides; and a 20-24 bp sequence complementary to the selected genomic locus. This first oligo was designed for each selected nucleic acid to provide a predicted uniform Tₘ with a two degree variation across all interrogations in the 480 assay set.

The second oligo used for each interrogation was a bridging oligo complementary to the genomic locus sequence directly adjacent to the genomic region complementary to the first oligonucleotide. Based on the selected nucleic acids of interest, the bridging oligos were designed to allow utilization of a total of 12 oligonucleotide sequences that could serve as bridging oligos for all of the 480 interrogations in the assay set.

The third oligo used for each interrogation was complementary to the 5′ region of the selected genomic locus, comprised the following sequential (5′ to 3′) elements: a 20-24 b sequence complimentary to the 5′ region in the genomic locus; a hybridization breaking nucleotide which was different from the corresponding base in the genomic locus; and a universal PCR priming sequence which is common to all third oligos in the assay set: ATTGCGGGGACCGATGATCGCGTC (SEQ ID NO:2). This third oligo was designed for each selected nucleic acid to provide a predicted uniform Tₘ with a two degree variation across all interrogations in the 480 assay set, and the Tₘ range was substantially the same as the Tₘ range as the first oligo set.

All oligonucleotides were synthesized using conventional solid-phase chemistry. The first and bridging oligonucleotides were synthesized with 5′ phosphate moieties to enable ligation to 3′ hydroxyl termini of adjacent oligonucleotides. An equimolar pool of sets of the first and third oligonucleotides used for all interrogations in the multiplexed assay was created, and a separate equimolar pool of all bridging oligonucleotides was created to allow for separate hybridization reactions.

Genomic DNA was isolated from 5 mL plasma using the Dynal Silane viral NA kit (Invitrogen, Carlsbad, Calif.). Approximately 12 ng DNA was processed from each of 37 females, including 7 non-pregnant female subjects, 10 female subjects pregnant with males, and 22 female subjects pregnant with females. The DNA was biotinylated using standard procedures, and the biotinylated DNA was immobilized on a solid surface coated with strepavidin to allow retention of the genomic DNA in subsequent assay steps.

The immobilized DNA was hybridized to the first pool comprising the first and third oligos for each interrogated sequences under stringent hybridization conditions. The unhybridized oligos in the pool were then washed from the surface of the solid support, and the immobilized DNA was hybridized to the pool comprising the bridging oligonucleotides under stringent hybridization conditions. Once the bridging oligonucleotides were allow to hybridize to the immobilized DNA, the remaining unbound oligos were washed from the surface and the three hybridized oligos bound to the selected nucleic acid regions were ligated using T4 ligase to provide a contiguous DNA template for amplification.

The ligated DNA was amplified from the solid substrate using an error correcting thermostable DNA polymerase, a first universal PCR primer TAATGATACGGCGACCACCGAGATCTACACCGGCGTTATGCGTCGAGA (SEQ ID NO:3) and a second universal PCR primer TCAAGCAGAAGACGGCATACGAGATXAAACGACGCGATCATCGGTCC CCGCAA (SEQ ID NO:4), where X represents one of 96 different sample indices used to uniquely identify individual samples prior to pooling and sequencing. 10 μL of universal PCR product from each of the 37 samples described above were and the pooled PCR product was purified using AMPure SPR1 beads (Beckman-Coulter, Danvers, Mass.), and quantified using Quant-iT™ PicoGreen, (Invitrogen, Carlsbad, Calif.).

The purified PCR product was sequenced on 6 lanes of a single slide on an Illumina HiSeq™ 2000. The sequencing run gave rise to 384M raw reads, of which 343M (89%) mapped to expected genomic loci, resulting in an average of 3.8 M reads per sample across the 37 samples, and 8K reads per sample per locus across the 480 loci. The mapped reads were parsed into sample and locus counts, and two separate metrics of percent fetal DNA were computed as follows.

Percent male DNA detected by chromosome Y loci corresponds to the relative proportion of reads derived from chromosome Y locus interrogations versus the relative proportion of reads derived from autosomal locus interrogations, and is computed as (number of chromosome Y reads in a test subject/number of autosome reads in test subject)/(number of reads in male control subject/number of autosome reads in the male control subject). This metric was used as a measure of percent fetal DNA in the case of a male fetus using the relative reads of chromosome Y.

Percent fetal DNA detected by polymorphic loci corresponds to the proportion of reads derived from non-maternal versus maternal alleles at loci where such a distinction can be made. First, for each identified locus, the number of reads for the allele with the fewest counts (the low frequency allele) was divided by the total number of reads to provide a minor allele frequency (MAF) for each locus. Then, loci with an MAF between 0.075% and 15% were identified as informative loci. The estimated percent fetal DNA for the sample was calculated as the mean of the minor allele frequency of the informative loci multiplied by two, i.e. computed as 2× average (MAF) occurrence where 0.075%<MAF<15%.

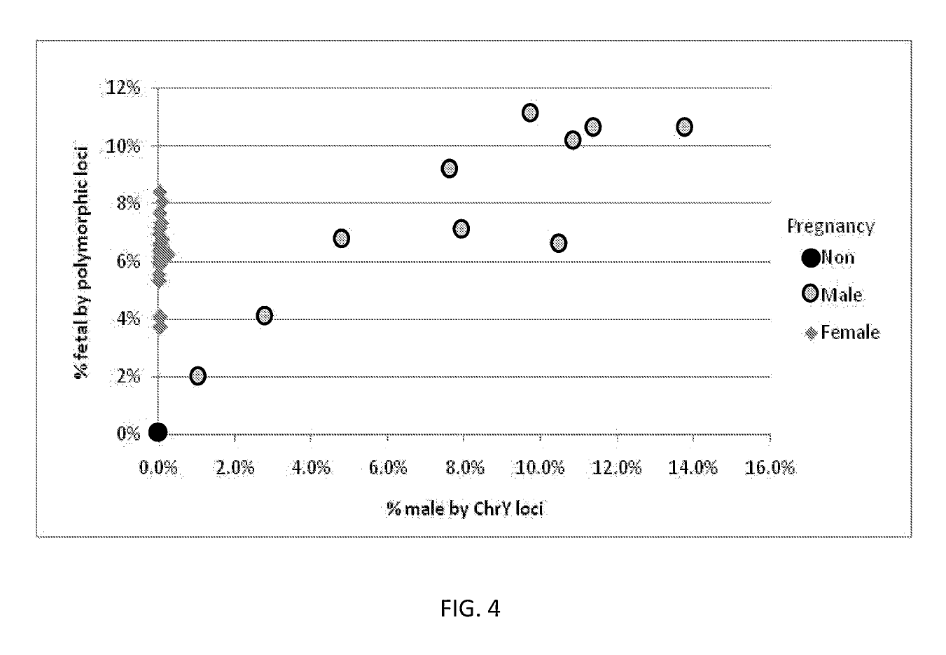

FIG. 4 demonstrates the results from these computations. As shown in FIG. 4, the percent male loci determined using the above-described chromosome Y metrics (grey circles) can separate pregnancies involving male fetuses from pregnancies involving female fetuses (grey diamonds) and non-pregnant samples (black circles). In addition, computation of the percent fetal amount in a sample by polymorphic loci metric can distinguish pregnant samples from non-pregnant samples. Finally, there is a correlation between the percent fetal DNA estimates for a sample obtained from chromosome Y and polymorphic loci in pregnancies involving male fetuses. This correlation persists down to quite low percent fetal values.

While this invention is satisfied by aspects in many different forms, as described in detail in connection with preferred aspects of the invention, it is understood that the present disclosure is to be considered as exemplary of the principles of the invention and is not intended to limit the invention to the specific aspects illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. §112, ¶6.

What is claimed is:

1. An assay system for providing a statistical likelihood of the presence or absence of a fetal aneuploidy comprising:
providing a maternal sample comprising maternal and fetal cell free DNA;
interrogating two or more polymorphic nucleic acid regions from a first chromosome;
detecting the interrogated polymorphic nucleic acid regions;
quantifying a relative frequency of alleles from the polymorphic nucleic acid regions from the first chromosome;
interrogating two or more selected nucleic acid regions from a second chromosome in the maternal sample;
detecting the interrogated two or more selected nucleic acid regions from the second chromosome;
quantifying a relative frequency of the interrogated two or more selected nucleic acid regions from the second chromosome;
comparing the relative frequency of the polymorphic nucleic acid regions from the first chromosome to the relative frequency of the polymorphic nucleic acid regions from the second chromosome;
calculating percent fetal cell free DNA in the maternal sample; and
providing a statistical likelihood of the presence or absence of a fetal aneuploidy using the calculated percent fetal cell free DNA in the maternal sample.

2. The assay system of claim 1, wherein at least the first chromosome is an autosome.

3. The assay system of claim 2, wherein both the first and second chromosomes are autosomes.

4. The assay system of claim 1, wherein calculating percent fetal cell free DNA comprises selecting polymorphic nucleic acid regions where maternal DNA is homozygous and fetal DNA is heterozygous.

5. The assay system of claim 4, wherein calculating percent fetal cell free DNA comprises computing a sum of low frequency alleles for the selected polymorphic nucleic acid regions.

6. The assay system of claim 1, wherein at least twenty-four polymorphic nucleic acid regions from the first chromosome and at least twenty-four polymorphic nucleic acid regions from the second chromosome are interrogated.

7. The assay system of claim 6, wherein at least forty-eight polymorphic nucleic acids from the first chromosome and at least forty-eight polymorphic nucleic acid regions from the second chromosome are interrogated.

8. The assay system of claim 7, wherein at least ninety-six polymorphic nucleic acids from the first chromosome and at least ninety-six polymorphic nucleic acid regions from the second chromosome are interrogated.

9. The assay system of claim 1, wherein an amplifying step is performed after one or both of the interrogating steps.

10. The assay system of claim 1, wherein an amplifying step is performed as part of one or both of the interrogating steps.

11. The assay system of claim 9, wherein the amplifying step is performed by PCR.

12. The assay system of claim 10, wherein the amplifying step is performed by PCR.

13. The assay system of claim 1, further comprising detecting levels of one or more non-maternally contributed region and quantifying a relative frequency of the one or more non-maternally contributed regions to determine the percent fetal cell free DNA in the maternal sample.

14. An assay system for providing a statistical likelihood of the presence or absence of a fetal aneuploidy comprising:
providing a maternal sample comprising maternal and fetal cell free DNA;
interrogating two or more polymorphic nucleic acid regions from a first chromosome;
detecting the interrogated polymorphic nucleic acid regions;
quantifying a relative frequency of alleles from the first chromosome;
selecting quantified polymorphic nucleic acid regions to identify low frequency and high frequency alleles on the first chromosome;
interrogating two or more polymorphic nucleic acid regions from a second chromosome;
detecting the interrogated polymorphic nucleic acid regions;
quantifying a relative frequency of alleles from the second chromosome;
selecting quantified polymorphic nucleic acid regions to identify low frequency and high frequency alleles on the second chromosome;
calculating the percent fetal cell free DNA using the selected high frequency alleles and low frequency alleles;
providing a statistical likelihood of the presence or absence of a fetal aneuploidy using the calculated percent fetal cell free DNA in the maternal sample.

15. The assay system of claim 14, wherein at least the first chromosome is an autosome.

16. The assay system of claim 15, wherein both the first and second chromosomes are autosomes.

17. The assay system of claim 14, wherein at least twenty-four polymorphic nucleic acid regions from the first chromosome and at least twenty-four polymorphic nucleic acid regions from the second chromosome are interrogated.

18. The assay system of claim 17, wherein at least forty-eight polymorphic nucleic acids from the first chromosome and at least forty-eight polymorphic nucleic acid regions from the second chromosome are interrogated.

19. The assay system of claim 18, wherein at least ninety-six polymorphic nucleic acids from the first chromosome and at least ninety-six polymorphic nucleic acid regions from the second chromosome are interrogated.

20. The assay system of claim 14, wherein an amplifying step is performed after one or both of the interrogating steps.

21. The assay system of claim 14, wherein an amplifying step is performed as part of one or both of the interrogating steps.

22. The assay system of claim 20, wherein the amplifying step is performed by PCR.

23. The assay system of claim 21, wherein the amplifying step is performed by PCR.
